(19) United States
(12) Patent Application Publication
(10) Pub. No.: US 2022/0104481 A1
(43) Pub. Date: Apr. 7, 2022

(54) CRYOPRESERVATION OF STEM CELLS

(71) Applicant: TiGenix, S.A.U., Madrid (ES)

(72) Inventors: Eleuterio LOMBARDO DE LA CAMARA, Madrid (ES); Maitane ORTIZ VIRUMBRALES, Madrid (ES)

(73) Assignee: TiGenix, S.A.U., Madrid (ES)

(21) Appl. No.: 17/430,623

(22) PCT Filed: Feb. 11, 2020
(86) PCT No.: PCT/EP2020/053440

§ 371 Date: Aug. 12, 2021

(30) Foreign Application Priority Data
Feb. 13, 2019 (EP) 19382099.0

(51) Int. Cl. A01N 1/02; C12N 5/0775; A61K 35/28

(52) U.S. Cl. CPC A01N 1/0221; C12N 2500/44; A61K 35/28; C12N 5/0667

(57) ABSTRACT

The invention relates to methods for the cryopreservation of a stem cell population, including mesenchymal stem cells (MSCs) such as adipose-derived stromal stem cells (ASCs). More particularly, the invention relates to the use of N-acetylcysteine (NAC) in cryopreservation methods, populations of cells obtained from said methods, compositions comprising said cells and uses thereof.

### TECHNICAL FIELD

The invention relates to methods for the cryopreservation of a stem cell population, including mesenchymal stem cells (MSCs) such as adipose-derived stromal stem cells (ASCs). More particularly, the invention relates to the use of N-acetylcysteine (NAC) in cryopreservation methods.

### BACKGROUND TO THE INVENTION

The global reparative and regenerative medicine marketplace requires that the viability and function of therapeutic cells is maintained, allowing transportation of cells from the place of manufacture to the patient, the completion of safety and quality control testing, and the formation of cell banks. Cells are either cryopreserved or hypothermically maintained before being returned to normothermic temperatures before or during utilisation. The success of these therapies depends, at least in part, on the ability to preserve not just the structure but also the function of the cells.

The goal of cell preservation, regardless of the type, is to halt biological time for a given period, followed by on-demand return of cellular viability, structure, and function. Ideally, the cell/tissue that is cryopreserved should have the same properties following thaw. The attainment of this goal is far from being realised in many cases. Preservation outcomes are often characterized by retention of a high degree of cell viability as measured immediately post-storage, followed by a subsequent decline over 24-48 hours coupled with a decrease in cellular responsiveness, function, and reproductive ability. For hypothermic preservation, storage intervals are typically limited to 1-3 days for most cellular systems.

Many studies have observed that cell properties (e.g. cellular activity, survival, proliferation potential) are affected by the freezing and thawing process. The preservation process places a number of stresses on cells as a result of temperature-dependent uncoupling of metabolic and biochemical processes. These include inter alia the production of free radicals by disruption of oxidative respiration, which are detrimental to cells due to the downstream effects of lipid peroxidation, DNA and RNA damage, cytoskeleton structural component alterations. Alterations in cellular membrane structure, fluidity, and organization can also activate membrane receptors, initiating a cascade of intracellular events including stimulation of stress-response pathways and apoptosis. Disregulation of cellular ionic balance through a shutdown of membrane-bound Na⁺/K⁺ pumps and Ca²⁺ ion channels activates stress-response mechanisms including the release of calcium from intracellular stores, osmotic influx, and cellular swelling. A host of additional stress response mechanisms can also be activated through low-temperature storage to the detriment of cells.

Cryoprotectants like dimethyl sulfoxide (DMSO), glycerol or animal-derived serum are commonly added to the cryopreservation medium to minimise these negative effects. However, there remains a need to improve methods of stem cell cryopreservation.

### SUMMARY OF THE INVENTION

The present invention is summarized as providing methods and compositions relating to stem cell cryopreservation, including mesenchymal stem cells (MSCs) such as adipose-derived stromal stem cells (ASCs), and uses of said compositions. In particular, to facilitate research studies and clinical applications of stem cells, the inventors have developed a novel cryopreservation approach that involves treating cells with N-acetylcysteine (NAC), which results in increased post-thaw viable cell number, increased growth rate, increased mitochondrial activity and/or improved recovery, while maintaining structural and/or functional properties of the cells, such as those required for their therapeutic use.

The invention provides a method for stem cell cryopreservation, the method comprising the steps of: (a) treating a population of stem cells with N-acetylcysteine (NAC) to obtain a treated population of stem cells; and (b) freezing the treated population of stem cells to obtain a frozen population of stem cells. In some embodiments, the method comprises the steps of: (a) treating the population of stem cells with NAC to obtain a treated population of stem cells; (b) freezing the treated population of stem cells to obtain a frozen population of stem cells; and (c) thawing the frozen population of stem cells to obtain a thawed population of stem cells. In some embodiments, the method comprises the steps of: (a) treating the population of stem cells with NAC to obtain a treated population of stem cells; (b) washing the treated population of stem cells to remove the NAC and to obtain a washed population of stem cells, and freezing the washed population of stem cells to obtain a frozen population of stem cells; and (c) thawing the frozen population of stem cells to obtain a thawed population of stem cells. In any of the methods, the treatment step may comprise incubating the population of stem cells with NAC for at least about 1, 2, 4, 6, 8, 10, 12, 16, 24 or 48 hours prior to freezing the population of stem cells. The treatment step may comprise adding NAC to the population of stem cells to an initial concentration in the range of around 0.5-10 mM. The treatment step may comprise one or more further additions of NAC to maintain the concentration of NAC at a preselected level. In some embodiments, the method further comprises the step of: (d) culturing the thawed population of stem cells to obtain an expanded population of stem cells. In some embodiments, the method further comprises the step of: (d) culturing the thawed population of stem cells in the presence NAC to obtain an expanded population of stem cells. The culturing step may comprise adding NAC to an initial concentration in the range of around 0.5-5 mM. The culturing step may comprise one or more further additions of NAC to maintain the concentration of NAC at a preselected level. In some embodiments, the method further comprises a step of washing the expanded population of stem cells to remove the NAC and to obtain a washed and expanded population of stem cells. In some embodiments, the method further comprises a step of washing the thawed population of stem cells or the expanded population of stem cells and resuspending the cells in a pharmaceutically acceptable carrier. In some embodiments, the method further comprises the step of: (e) freezing the expanded or the washed and expanded population of stem cells to obtain a frozen expanded population of stem cells or a frozen, washed and expanded population of stem cells. In some embodiments, the method further comprises the steps of: (e) freezing the expanded or the washed and expanded population of stem cells to obtain a frozen expanded population of stem cells or a frozen, washed and expanded population of stem cells; and (f) thawing the frozen expanded or the frozen, washed and expanded population of stem cells to obtain a thawed expanded population of stem cells. In some embodiments, the method further comprises the step of: (g) washing the thawed expanded population of stem cells and resuspending the cells in a pharmaceutically acceptable carrier.

The invention also provides a method for stem cell cryopreservation, the method comprising the steps of: (a) freezing a population of stem cells to obtain a frozen population of stem cells; (b) thawing the frozen population of stem cells to obtain a thawed population of stem cells; and (c) culturing the thawed population of stem cells in the presence NAC to obtain an expanded population of stem cells. The culturing step may comprise adding NAC to an initial concentration of around 0.5-5 mM. In some embodiments, the culturing step comprises one or more further additions of NAC to maintain the concentration of NAC at a preselected level.

In any of the methods of the invention, the freezing step may comprise reducing the temperature to between −70° C. and −130° C. at a rate of between about −0.5 to about −10° C./minute. In some embodiments, the freezing step comprises reducing the temperature from +4° C. to between −100 and −180° C. in 10-60 mins.

In any of the methods of the invention, the population of stem cells may be thawed at 37° C. The cell density of the frozen population of stem cells may be in the range of around 1 million to around 50 million cells/mL, preferably around 25 million cells/mL.

In some embodiments, the population of stem cells is substantially pure. In some embodiments, the stem cells are mesenchymal stem cells (MSCs). In some embodiments, the stem cells are adipose-derived stromal stem cells (ASCs). In some embodiments, the stem cells are human cells. In preferred embodiments, the stem cells are human ASCs.

In any of the methods of the invention, the method may further comprise the step of resuspending the cells in a pharmaceutically acceptable carrier. The method may comprise freezing the population of stem cells in a plurality of cryovials.

In some embodiments, the method comprises repeating the steps of any one of methods of the invention for a plurality of populations of stem cells. The method may comprise freezing the plurality of populations of stem cells in a plurality of cryovials. The method may further comprise storing the plurality of cryopreservation vials in a liquid nitrogen storage container for at least one month at least 2 months, at least 3 months, at least 6 months, or at least 1 year.

The invention further provides a liquid nitrogen storage container containing a plurality of cryopreservation vials obtained according to a method of the invention.

The invention provides a population of stem cells obtained by a method of the invention.

In any of the methods of the invention or population of stem cells of the invention, the number of viable cells following thaw and optionally culture for about 1 day and/or about 4 days may be increased as compared to a control population of stem cells. In any of the methods of the invention or population of stem cells of the invention, the number of viable cells following thaw may be increased at least about 1.05-fold, at least about 1.1-fold, at least about 1.2-fold, at least about 1.3-fold, at least about 1.4-fold, at least about 1.5-fold, at least about 1.6-fold, at least about 2-fold, or at least about 5-fold as compared to a control population of stem cells. In any of the methods of the invention or population of stem cells of the invention, the growth rate following thaw may be increased at least about at least about 1.03-fold, 1.05-fold, at least about 1.1-fold, at least about 1.15-fold, at least about 1.2-fold, at least about 1.25-fold, at least about 1.3-fold, at least about 1.4-fold, at least about 1.6-fold, or at least about 2-fold in the population of stem cells as compared to a control population of stem cells. In any of the methods of the invention or population of stem cells of the invention, mitochondrial activity following thaw and optionally culture for about 1 day and/or about 4 days may be increased at least about 5%, at least about 10%, at least about 15%, at least about 20%, at least about 30%, at least about 35%, at least about 40% or at least about 50% as compared to a control population of stem cells. In any of the methods of the invention or population of stem cells of the invention, the time taken post-thaw for the ASCs to recover may be decreased as compared to a control population of stem cells. In any of the methods of the invention or population of stem cells of the invention, the number of hours taken for the cells to recover post-thaw may be decreased at least about 1.1-fold, at least about 1.2-fold, at least about 1.4-fold, at least about 1.6-fold, at least about 2-fold, at least about 3-fold, at least about 4-fold, or at least about 5-fold relative to a control population of stem cells.

The invention provides a cryopreservation composition comprising the population of stem cells of the invention and a cryopreservation medium. The composition may be frozen. In some embodiments, the composition contains NAC.

The invention also provides a pharmaceutical composition comprising the population of stem cells of the invention and a pharmaceutically acceptable carrier. The composition may comprise around 1 million cells to around 150 million cells, preferably around 30 million cells or around 120 million cells. In some embodiment, the cell density is around 1 to 20 million cells/mL. The invention provides the use of NAC for the cryopreservation of stem cells, e.g. in a method of the invention.

The invention also provides a population of stem cells of the invention, pharmaceutical composition of the invention or cryopreservation composition of the invention for use in therapy.

The invention further provides the population of stem cells of the invention, pharmaceutical composition of the invention or cryopreservation composition of the invention for use in a method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection in a patient in need thereof.

The invention provides a method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection, the method comprising administering the population of stem cells of the invention, pharmaceutical composition of the invention or cryopreservation composition of the invention to a subject in need thereof.

The invention also provides a population of stem cells for use in a method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection, in a patient in need thereof, wherein the method comprises the steps of: (a) treating of a population of stem cells with NAC to obtain a treated population of stem cells; (b) freezing the treated population of stem cells to obtain a frozen population of stem cells; (c) thawing the frozen population of stem cells to obtain a thawed population of stem cells; (d) optionally culturing the thawed population of stem cells to obtain an expanded population of stem cells; and (e) administering the population of stem cells to the patient.

The invention further provides a method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection, in a patient in need thereof, the method comprising the steps of: (a) treating a population of stem cells with NAC to obtain a treated population of stem cells; (b) freezing the treated population of stem cells to obtain a frozen population of stem cells; (c) thawing the frozen population of stem cells to obtain a thawed population of stem cells; (d) optionally culturing the thawed population of stem cells to obtain an expanded population of stem cells; and (e) administering the population of stem cells to the patient.

The invention provides a population of stem cells for use in a method of treating fistula and/or treating and/or preventing an inflammatory disorder, an autoimmune disease or an immunologically-mediated disease, such as sepsis, rheumatoid arthritis, allergies (e.g. hypersensitivity Type IV reactions), irritable bowel disease, Crohn's disease, ulcerative colitis or organ rejection in a patient in need thereof, wherein the method comprises the steps of: (a) freezing a population of stem cells to obtain a frozen population of stem cells; (b) thawing the frozen population of stem cells to obtain a thawed population of stem cells; (c) culturing the thawed population of stem cells in the presence NAC to obtain an expanded population of stem cells; and (d) administering the population of stem cells to the patient.

The invention also provides a method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection, in a patient in need thereof, the method comprising the steps of: (a) freezing a population of stem cells to obtain a frozen population of stem cells; (b) thawing the frozen population of stem cells to obtain a thawed population of stem cells; (c) culturing the thawed population of stem cells in the presence NAC to obtain an expanded population of stem cells; and (d) administering the population of stem cells to the patient.

In some embodiments, the population of stem cells for use according to the invention or method of treatment according to the invention further comprises any one of the steps of the methods of stem cell cryopreservation described herein prior to administration of the population of stem cells to the patient.

In some embodiments of the population of stem cells, pharmaceutical composition or cryopreservation composition for use according to the invention, or method of treatment of the invention, the method comprises administering around 1 million to 150 million cells, preferably around 30 million stem cells or around 120 million stem cells. The method may comprise administering around 1 million to around 10 million cells/kg. The method may comprise injecting the population of stem cells, pharmaceutical composition or cryopreservation composition of the invention. The stem cells may be as defined herein. In some embodiments, the stem cells are allogeneic or autologous. In preferred embodiments, the stem cells are human, allogeneic ASCs.

The invention provides a cryopreservation kit comprising: a cryovial, a container containing NAC and a container comprising a population of stem cells.

### BRIEF DESCRIPTION OF THE FIGURES

FIG. 1. Flowchart illustrating the exemplified assays.

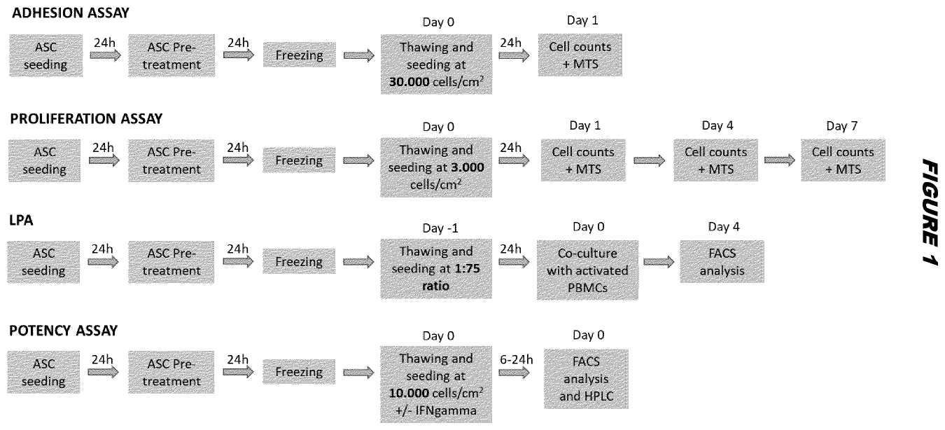

FIG. 2. MTS assay at 24 hours after post-thaw seeding of ASCs that have been treated with various compounds prior to freezing (NAC; LY294,002, sc-79 or exendin-4), as compared to non-treated (NT) cells. Data representative of a single experiment in technical six technical repeats for MTS.

FIG. 3. Cell numbers at 24 hour after post-thaw seeding of ASCs that have been treated prior to freezing with 6 mM NAC (NAC), as compared to non-treated (NT) cells. Data representative of a single experiment in technical triplicates.

FIG. 4. Cell density at 1, 4, and 7 days (A) and MTS assay at 24 hours (B) and 96 hours (C), after post-thaw seeding of ASCs that have been treated prior to freezing with 6 mM NAC (NAC), as compared to non-treated (NT) cells. MTS results are presented as percentage of absorbance at 490 nm relative to the non-treated cells. Data representative of a single experiment in triplicates for cell counts, and in 6 technical repeats for MTS. The 0 day time point in FIG. 4A shows the cell seeding density, rather than number of viable adhered cells as shown for the other time points.

FIG. 5. Graph showing cell densities of ASCs from two different donors (donor A (DON A) and donor B (DON B)) at 1, 4 and 7 days after post-thaw seeding. ASCs were pretreated with 6 mM NAC and compared to non-treated cells. Data representative of one experiment in technical triplicates.

FIG. 6. Graph showing cell densities at 7, 11 and 14 days after seeding of thawed ASC with post-thaw treatment with 2, 6 or 12 mM NAC added to the plating medium. Data representative of two experiments in technical triplicates.

FIG. 7. ASC identity assay by flow cytometry. ASCs (from donor A and treated prior to freezing with 6 mM NAC) were analysed two weeks after thawing and compared to non-treated cells for CD29, CD73, CD90 and CD105. The percentages of positive cells are shown in the figure. Experiment run in technical triplicates.

FIG. 8. Lymphocyte proliferation assay using thawed ASCs from donor A pretreated with 6 mM NAC and compared to non-treated cells. The analysis was performed at 96 hours using a ratio for ASC:PBMC of 1:75. (A) Overlays between the maximal proliferation of activated PBMCs and the PBMCs in the presence of ASC. (B) Comparison between NAC treated and non-treated ASCs post-thaw on lymphoproliferation. The results are quantified in the bottom right panel.

FIG. 9. Diagram showing the planning and timing of ASC and monocytes co-cultures, and the analysis performed to assess the effect of ASC on macrophage and mDC differentiation and function

FIG. 10. Microscopy images at 2× of mature DC cultures alone or in the presence of thawed ASCs from two different donors (donor A (DON A) and donor B (DON B)) pretreated with NAC or non-treated.

FIG. 11. Microscopy images at 20× of mature DC cultures alone or in the presence of thawed ASCs from two different donors (donor A (DON A) and donor B (DON B)) pretreated with NAC or non-treated.

FIG. 12. Histograms representing the phagocytosis of Staphylococcus aureus particles by mDC in the absence or presence of ASCs from two different donors (donor A (DON A) and donor B (DON B)) with or without NAC pretreatment, measured by flow cytometry.

FIG. 13. Surface expression of the phagocytic receptor CD206 (mannose receptor) by mDC in the absence or presence of ASCs from two different donors (donor A (DON A) and donor B (DON B)) with or without NAC pre-treatment, measured by flow cytometry. ASC induce the expression of CD14, CD206 and CD163 in mDC. ASC NAC pretreatment did not alter these effects.

FIG. 14. Surface expression of the phagocytic receptor CD163 (scavenger receptor) by mDC in the absence or presence of ASCs from two different donors (donor A (DON A) and donor B (DON B)) with or without NAC pre-treatment, measured by flow cytometry. ASC induce the expression of CD14, CD206 and CD163 in mDC. ASC NAC pretreatment did not alter these effects.

FIG. 15. Dot plots representing the surface expression of CD14 and CD1a (antigen presenting molecule) by mDC in the absence or presence of ASCs from two different donors (donor A (DON A) and donor B (DON B)) with or without NAC pretreatment, measured by flow cytometry. mDC are CD14-CD1a+, but the presence of ASC generates a new modulatory CD14+CD1a− DC population. ASC NAC pretreatment did not modify this effect.

### DETAILED DESCRIPTION

The present invention relates to methods and compositions for stem cell cryopreservation, where a population of stem cells is treated with N-acetylcysteine (NAC) prior to freezing (“NAC pretreatment”) and/or after the stem cells are thawed (“post-thaw treatment”).

The inventors tested a number of compounds that are known to modulate apoptotic insults in cells (such as hypoxia, serum deprivation, oxidative stress (e.g. caused by hydrogen peroxide treatment), Fas ligand induced death etc.) with the aim of improving the resistance of cells to the freeze-thaw process. NAC was found to confer an advantage to the stem cells post-thaw in terms of increasing viable cell number, increasing growth rate, increasing mitochondrial activity and/or improving recovery compared to non-treated control cells. Increasing the number of viable cells available immediately upon thaw is useful, e.g. for acute treatment. These benefits will help to facilitate storage, shipping and handling of stem cell stocks and cell lines, and the preparation and shipment of cell-based therapies, e.g. by decreasing the time required to recover and/or expand cryopreserved cells in culture after thaw.

### N-Acetylcysteine

N-Acetylcysteine (NAC), also known as N-acetyl-L-cysteine, is the nonproprietary name for the N-acetyl derivative of the naturally occurring amino acid, L-cysteine. It is an antioxidant having a molecular weight of 163.2 gmol⁻¹ and the following chemical structure:

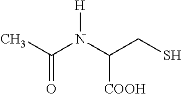

NAC is marketed under the trade names of Acetadote®, Mucomyst®, Parvolex®, Fluimucil®, and others. It is approved for several indications including treatment of paracetamol (acetaminophen) overdose (as an injectable and an oral agent), and as a mucolytic to loosen thick mucus in individuals with cystic fibrosis or chronic obstructive pulmonary disease (taken intravenously, by mouth or inhaled as mist). NAC is also being used or investigated to treat other indications including liver failure, various cancers, methacrylonitrile poisoning, reduction of radio contrast-induced nephropathy, and reduction of reperfusion injury during cardio bypass surgery.

Pretreatment with NAC

Disclosed herein is a method for stem cell cryopreservation, the method comprising the treatment of a population of stem cells with NAC prior to freezing i.e. “pretreatment” of a population of stem cells. Thus, “NAC pretreated cells” refers to cells that have been treated with NAC and then frozen.

The method for stem cell cryopreservation may comprise the steps of: (a) treating a population of stem cells (such as ASCs) with N-acetylcysteine to obtain a treated population of stem cells; and (b) freezing the treated population of stem cells to obtain a frozen population of stem cells.

Treating the population of stem cells with NAC (the “treatment” or “treatment step”) is typically carried out by adding NAC to a suitable cell culture medium for the population of stem cells. A stock solution of NAC can be prepared, for example in water, and then the NAC can be diluted to the required concentration in the culture medium.

The skilled person will be aware of suitable cell culture media for supporting the growth of particular cell types. Cell culture media can be in liquid or solid form, including gelatinous media such as agar, agarose, gelatin and collagen matrices. A medium can be “defined medium” that are made of chemically defined (usually purified) components, and that do not contain poorly characterized biological extracts such as yeast extract and beef broth. A medium can be a “basal medium” which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. Examples of basal media include, but are not limited to, Eagle's Basal Medium, Minimum Essential Medium, Dulbecco's Modified Eagle's Medium (DMEM), Medium 199, Nutrient Mixtures Ham's F-10 and Ham's F-12, McCoy's 5A, Dulbecco's MEM/F-12, alpha modified Minimal Essential Medium (alphaMEM), Roswell Park Memorial Institute Media 1640 (RPMI 1640), and Iscove's Modified Dulbecco's Medium (IMDM). Typically, 0 to 20% Fetal Bovine Serum (FBS) or 1-20% horse serum will be added to the above media in order to support the growth of MSCs. However, a defined medium could be used if the necessary growth factors, cytokines, and hormones in FBS for MSCs are identified and provided at appropriate concentrations in the growth medium. Antibiotics which can be included in the culture medium include, but are not limited to penicillin and streptomycin. The concentration of penicillin in the chemically defined culture medium is about 10 to about 200 units per ml. The concentration of streptomycin in the chemically defined culture medium is about 10 to about 200 μg/ml. For example, a suitable cell culture medium for ASCs is complete DMEM (DMEM/F-12 media—GlutaMAX™-I, Gibco, supplemented with 100 μg/mL penicillin/streptomycin and 10% FBS). The treatment step may comprise adding NAC to the population of stem cells to an initial concentration in the range of around 0.5-10 mM NAC, for example, around 2-8 mM or around 4-6 mM. An initial concentration of 0.5-20 mM NAC may also be used, for example, around 3-15 mM NAC, 0.5-12 mM or 4-12 mM NAC. In a particularly preferred embodiment, the initial concentration of NAC is around 6 mM. The “initial concentration” refers to the concentration of NAC when added to the population of stem cells. However, it will be understood that after addition to the cells, the initial concentration of NAC will likely decrease, e.g. by NAC being degraded or metabolised. Thus, the treatment step may comprise one or more further additions of NAC, for example, to maintain the concentration of NAC to which the population of stem cells is exposed. Thus, the “treatment step” may comprise treating of the population of stem cells with an initial concentration of NAC, optionally monitoring the level of NAC during the treatment step, and adding one or more further additions of NAC to maintain the concentration of NAC the initial concentration or a preselected level (e.g. a concentration of NAC described above).

The treatment step may comprise incubating the population of stem cells with NAC for at least about 1, 2, 4, 6, 8, 10, 12, 16, 24 or 48 hours prior to freezing the population of stem cells. For example, the incubation of the population of stem cells with NAC may be carried for between about 1 and about 48 hours, between about 2 and 24 hours, or between about 6 and 24 hours prior to freezing the population of stem cells. The incubation may be carried out under any suitable conditions (e.g. where the population of stem cells are stable). In preferred embodiments, the incubation is carried out under culture conditions for the particular cell type. For example, ASCs can be incubated with NAC in complete DMEM (DMEM/F-12 media—GlutaMAX™-I, Gibco, supplemented with 100 μg/mL penicillin/streptomycin and 10% FBS) and incubated at 37° C. at 5% CO₂. In one embodiment, the population of stem cells is not incubated with NAC for the whole culture period. The culture period is the period between seeding the population of stem cells in a cell culture vessel and freezing the population of stem cells. In one embodiment, the population of stem cells is incubated in a culture medium without added NAC for a first period and then incubated in a culture medium with added NAC for a second period.

A population of stem cells that has been subjected to a NAC “treatment step” as disclosed herein is referred to as a “treated population of stem cells”.

Following the treatment step, the treated population of stem cells is frozen. A population of stem cells that has been subjected to freezing (a “freezing step”) as disclosed herein is referred to “a frozen population of stem cells”. A population of stem cells that has been subjected to thawing (a “thawing step”) as disclosed herein is referred to “a thawed population of stem cells”. Thus, the method may comprise the steps of: (a) treating the population of stem cells with NAC to obtain a treated population of stem cells; (b) freezing the treated population of stem cells to obtain a frozen population of stem cells; and (c) thawing the frozen population of stem cells to obtain a thawed population of stem cells.

Before the treated population of stem cells are frozen, the NAC may be removed (i.e. so the cells are no longer exposed to extracellular NAC). Typically, this can be carried out by washing the population of stem cells, for example, with (1) a cell culture medium (e.g. as used in the treatment step) that does not contain NAC; (2) phosphate buffered saline (PBS); and/or (3) a freezing medium. A population of stem cells that has been subjected to washing (a “washing step”) as disclosed herein is referred to “a washed population of stem cells”. Washing can also be used as a medium exchange step so that the cells can be frozen in a different medium, such as freezing medium. Thus, the method may comprise the steps of: (a) treatment of the population of stem cells with N-acetylcysteine to obtain a treated population of stem cells; (b) washing the treated population of stem cells to remove the N-acetylcysteine and to obtain a washed population of stem cells, and freezing the washed population of stem cells to obtain a frozen population of stem cells; and (c) thawing the frozen population of stem cells to obtain a thawed population of stem cells.

Washing the treated population of stem cells can be carried out by any suitable method. For adherent cells, the NAC containing solution (e.g. medium) can be exchanged for a different one (e.g. that does not contain NAC and/or is a freezing medium) by simple pipetting. For cells in suspension (including trypsinized adherent cells), the cells can be pelleted, e.g. using a centrifuge, the supernatant removed, optionally washed (e.g. with a culture medium or PBS) and then resuspended in the required medium (e.g. a culture medium or freezing medium). Filtration, ultrafiltration or dialysis can also be used to wash the cells. Methods for trypsinizing adherent cells are known in the art and a suitable method is exemplified in the examples.

Following freeze-thaw, the cells can be cultured (“culturing” or a “culturing step”), e.g. to allow the cells to recover and/or to increase cell number. The resulting cells are termed an “expanded population of stem cells”. The term “expanded” as used herein when referring to cells shall be taken to have its usual meaning in the art, namely cells that have been proliferated in vitro. “Proliferation” refers to an increase in cell number. “Proliferating” and “proliferation” refer to cells undergoing mitosis. Thus, the method may further comprise the step of: (d) culturing the thawed population of stem cells to obtain an expanded population of stem cells.

“Culturing” as used herein refers to the term as recognized in the art, namely any method of achieving cell growth in a suitable medium. Cells may be cultured by any technique known in the art for the culturing of stem cells. The culturing step can be small scale, medium scale or large scale. A culture can be considered small scale if the total culture volume is less than about 100 mL. A culture can be considered medium scale if the total culture volume is between about 100 mL and about 5 L. A culture can be considered large scale if the total culture volume (e.g. in a bioreactor) is greater than about 5 L, and may be greater than 10 L, 100 L, 500 L or 1000 L. A “cell culture” refers to a growth of cells in vitro. In such a culture, the cells proliferate, but they do not organize into tissue per se. A “tissue culture” refers to the maintenance or growth of tissue, e.g., explants of organ primordial or of an adult organ in vitro so as to preserve its architecture and function. A “monolayer culture” refers to a culture in which cells multiply in a suitable medium while mainly attached to each other and to a substrate. Furthermore, a “suspension culture” refers to a culture in which cells multiply while suspended in a suitable medium. Likewise, a “continuous flow culture” refers to the cultivation of cells or explants in a continuous flow of fresh medium to maintain cell growth, e.g. viability. A “confluent culture” is a cell culture in which all the cells are in contact and thus the entire surface of the culture vessel is covered, and implies that the cells have also reached their maximum density, though confluence does not necessarily mean that division will cease or that the population will not increase in size.

A discussion of various culture techniques, as well as their scale-up, may be found in Freshney, R. I., Culture of Animal Cells: A Manual of Basic Technique and Specialized Applications, 7th Edition, Wiley-Blackwell January 2016. The culturing step may be carried out in any type of vessel (for a review of the manufacture of MSCs, including a discussion of different types of vessel see Mizukami et al. “Mesenchymal Stromal Cells: From Discovery to Manufacturing and Commercialization” Stem Cells International (2018) Article ID 4083921, 1-13 https://doi.org/10.1155/2018/4083921). Examples of vessels that can be used in the methods disclosed herein include monolayer culture or flat two-dimensional flasks, which consist of a single compartments or multi-layered vessel cell factories such as Nunc Cell Factories and Corning Cell Stacks. As an alternative to flasks, roller bottles can be used, i.e. cylindrical bottles place into a rotating apparatus in which the cells can form a monolayer on around the inner surface of the bottle. Bioreactors suitable for the large-scale expansion of cells, including MSCs (such as ASCs), are commercially available and may include both 2D (i.e. substantially planar) and 3D expansion bioreactors. Examples of such bioreactors that may be used in the methods disclosed herein include, but are not limited to, a plug flow bioreactor, a perfusion bioreactor, a continuous stirred tank bioreactor, or a stationary-bed bioreactor. The bioreactor can be operated in batch, fed-batch or perfusion mode. Due to anchorage-dependent nature of MSCs, culturing in bioreactors requires the use of a microcarrier, which are generally small beads (100-200 μm in dimeter) that are easily maintained in suspension and provide a surface for the cells to attach and grow. Examples of microcarriers include the Cytodex-3 microcarrier (GE Healthcare). Cells are typically grown at temperatures between 31° C. to 37° C. in a humidified environment. Thus, in some embodiments, culture of the thawed population of stem cells (e.g. MSCs, such as ASCs) to obtain an expanded population of stem cells is carried out in a large scale bioreactor using a microcarrier.

Culturing of the thawed population of stem cells may be carried out in the presence of NAC, e.g. to improve recovery and/or to increase cell number. In other words, post-thaw NAC treatment can be used in addition to pretreatment with NAC. Thus, the method may further comprise the step of: (d) culturing the thawed population of stem cells in the presence N-acetylcysteine to obtain an expanded population of stem cells. Culturing the thawed population of stem cells may comprise adding NAC to an initial concentration in the range of around 0.5-5 mM NAC, such as around 0.5-4 mM or around 1-2 mM, preferably around 2 mM, under suitable cell culture conditions for the cell type. Further additions of NAC may be required to maintain the concentration of NAC in the cell culture medium (e.g. due to NAC being degraded or metabolised). Thus, the culturing step may comprise adding an initial concentration of NAC in the culture medium, followed by further additions to NAC to maintain the initial concentration of NAC or to maintain the concentration of NAC at a preselected level (e.g. a concentration of NAC as described above). Further additions can be added as a bolus of NAC alone or in combination with other nutrients (e.g. in fed-batch culture). The “culturing step” may further comprise monitoring the level of NAC, and adding one or more further additions of NAC to maintain the initial concentration or a preselected level. Alternatively, NAC can be continuously supplemented, e.g. in the fresh media during perfusion culture.

NAC can be removed prior to any downstream uses of the population of stem cells if required. Thus, the method may further comprise a step of washing the expanded population of stem cells to remove the NAC and to obtain a washed and expanded population of stem cells. The washing step can allow medium exchange e.g. into a pharmaceutically acceptable carrier, a solution/medium that does not contain NAC or a freezing medium. Washing can be carried out by any suitable method, including centrifugation, filtration, ultrafiltration or dialysis. For adherent cells, the NAC containing solution (e.g. medium) can be exchanged for a different one by simple pipetting. For cells in suspension (including trypsinized adherent cells), the cells can be pelleted (e.g. using a centrifuge), the supernatant removed, optionally washed (e.g. with a culture medium or PBS) and then resuspended in the required solution (e.g. a culture medium, a freezing medium or a pharmaceutically acceptable carrier). Thus, the method may further comprise a step of washing the thawed or expanded population of stem cells (e.g. of step (c) or (d)) and resuspending the cells (e.g. suspension cells or trypsinized adherent cells) in a pharmaceutically acceptable carrier.

The expanded population of stem cells may be frozen, e.g. for storage as a cell stock and/or for shipping. The method may further comprise the step of: (e) freezing the expanded population of stem cells (e.g. from step (d)) to obtain a frozen expanded population of stem cells. The method may further comprise the steps of: (e) freezing the expanded population of stem cells to obtain a frozen expanded population of stem cells; and (f) thawing the frozen expanded population of stem cells to obtain a thawed expanded population of stem cells. The method may comprise the step of: (e) freezing the washed and expanded population of stem cells to obtain a frozen, washed and expanded population of stem cells. The method may further comprise the steps of: (e) freezing the washed and expanded population of stem cells to obtain a frozen, washed and expanded population of stem cells; and (f) thawing the frozen, washed and expanded population of stem cells to obtain a thawed expanded population of stem cells. As the “culturing step” of step (d) can be carried out in the presence of NAC as discussed above, in these instances, the expanded population of stem cells may be considered “pretreated” with NAC prior to freezing. The NAC can be removed by washing, if required, prior to freezing and/or washing can be used for medium exchange e.g. into a freezing medium. Optionally, the method may further comprise the step of: (g) washing the thawed expanded population of stem cells and resuspending the cells (e.g. the suspension or trypinized adherent cells) in a pharmaceutically acceptable carrier.

The frozen population of stem cells (e.g. ASCs) obtained from the methods discussed above form a master cell stock. For example, the population of stem cells can be aliquoted into a plurality of cryovials, e.g. at least about 10, at least about 20, at least about 50, about 100, about 1000, about 2000, about 5000 or more cryovials and stored cryogenically (e.g. in a liquid nitrogen storage container). Individual cryovials can then be thawed separately for downstream uses. The thawed or expanded population of stem cells (e.g. ASCs) obtained from the methods discussed above may be a therapeutic stem cell population. For example, the thawed or expanded population of stem cells (e.g. ASCs) may be in a suitable formulation (e.g. a pharmaceutical composition containing a pharmaceutically acceptable carrier) for administration to a patient in need thereof.

The method may further comprise the step of resuspending the cells in a pharmaceutically acceptable carrier.

### Post-Thaw NAC Treatment

Disclosed herein is a method for stem cell cryopreservation, the method comprising the steps of: (a) freezing a population of stem cells (such as ASCs) to obtain a frozen population of stem cells; (b) thawing the frozen population of stem cells to obtain a thawed population of stem cells; and (c) culturing the thawed population of stem cells in the presence NAC to obtain an expanded population of stem cells. Culturing the thawed population of stem cells in the presence of NAC (i.e. post-thaw NAC treatment) may improve recovery and/or increase viable cell number.

Culturing the thawed population of stem cells may comprise adding NAC to an initial concentration in the range of around 0.5-5 mM NAC, such as around 0.5-4 mM or around 1-2 mM, preferably around 2 mM, under suitable cell culture conditions for the cell type. Further additions of NAC may be required to maintain the concentration of NAC in the cell culture medium (e.g. due to NAC being degraded or metabolised). Thus, the culturing step may comprise adding an initial concentration of NAC in the culture medium, followed by further additions to NAC to maintain the initial concentration of NAC or to maintain the concentration of NAC at a preselected level (e.g. a concentration of NAC for post-thaw treatment as described above). Further additions can be added as a bolus, optionally in combination with other nutrients (e.g. in fed-batch culture). The “culturing step” may further comprise monitoring the level of NAC, and adding one or more further additions of NAC to maintain the initial concentration or a preselected level. Alternatively, NAC can be continuously supplemented, e.g. in the fresh media provided during perfusion culture.

The method may further comprise the step of resuspending the cells in a pharmaceutically acceptable carrier.

### Cryopreservation

Herein, the term “cryopreservation” is used to describe the storage of cells in low temperature environments, i.e. −70° C. to −196° C. These temperatures are suitable for long term storage (months to years). The use of the terms “freezing”, to “freeze” and “frozen” in the context of stem cells as discussed herein refers to the act of exposing the cells to, and cells that have been subjected to, such low temperatures.

Typically, upon cooling, as the external medium freezes, cells equilibrate by losing water, thus increasing intracellular solute concentration. Below about −10 to −15° C. intracellular freezing will occur. Both intracellular freezing and solution effects are responsible for cell injury. Physical damage by extracellular ice is largely a result of plasma membrane injury resulting from osmotic dehydration of the cell.

Not all biological processes halt once a system is frozen. During freezing, cells remain in a biochemically active unfrozen state while encased in a frozen ice matrix. Not until temperatures drop below the glass transition point (T_{g}) of the cryoprotectant/cell solution mixture (typically below −100° C.) will cells enter a glassy state, in which biochemical and biomolecular activity cease.

During freezing and subsequent thawing, when temperatures are above T_{g} a significant set of molecular and biochemical events occur within each cell that drastically influence its post-thaw viability and function. In this temperature range (from around +15° C. to −99.9° C.) a number of similarities can be seen in cellular response mechanisms between cryopreservation and hypothermic storage. Such events include the formation of free radicals, uncoupling of biochemical pathways, intracellular waste accumulation, ion-gradient disruption, protein denaturation and degradation, and enzyme cleavage and activation. These and other events can activate apoptotic and/or necrotic cell death pathways, which can result in the phenomenon of delayed-onset cell death. This can be observed as a disconnect between the measure of viability immediately post storage and true survival 24-48 hours later.

### Cryopreservation Medium

The population of stem cells (such as ASCs) may be frozen in a cryopreservation medium (a “freezing medium”). The medium may preserve (to a certain extent) one or more of the properties of the cells (e.g. viability) following freeze-thaw and/or may aid recovery. The cryopreservation medium may contain NAC, e.g. at a concentration of between about 0.5-10 mM. In one embodiment, the cryopreservation medium does not contain NAC. A cryopreservation medium generally contains one or more cryopreservation agents such as DMSO, PVP, sericin, or methylcellulose, and/or may contain a commercially available cryopreservation solution. The one or more cryopreservation agents or cryopreservation solution may be added to the stem cell culture medium, such as DMEM, to produce a cryopreservation medium. In one embodiment, the cryopreservation medium does not contain any added growth factor. In one embodiment, the cryopreservation medium does not contain any added EGF and bFGF. In one embodiment, the cryopreservation medium does not contain added sodium selenite. In one embodiment, the cryopreservation medium does not contain NAC and does not contain any added growth factor. In one embodiment, the cryopreservation medium does not contain NAC and does not contain any added EGF and bFGF. In one embodiment, the cryopreservation medium does not contain NAC and does not contain any added sodium selenite. In one embodiment, the cryopreservation medium does not contain NAC and does not contain any added growth factor and does not contain any added sodium selenite. In one embodiment, the cryopreservation medium does not contain NAC and does not contain EGF and bFGF and does not contain any added sodium selenite.

A cryopreservation agent (or cryoprotectant) is ideally nontoxic, protects cells during freezing, substitutes for water and/or has a high glass transition temperature. Without wishing to be bound by theory, cryoprotectants are hypothesized to protect cells from freezing through, inter alia, the following mechanisms: counterbalancing external osmotic pressure, stabilizing biomolecules via preferential exclusion, forming a protective glass around biological molecules, and preventing damaging phase transitions in lipid membranes.

Historically, DMSO, glycerol and animal serum have been used as cryoprotectants.

DMSO is typically added to a cryopreservation medium in the range of 1-20% (v/v), such as 5-15%, i.e. about 1%, 2%, 5%, 10% or 20%. A final concentration of around 10% is particularly preferred.

DMSO may be used in combination with serum, i.e. fetal calf/bovine serum (FCS/FBS) or human serum. For example, the cryopreservation medium may contain 20-95% serum (human or FCS) and 5-15% DMSO. A particularly preferred cryopreservation medium (e.g. for MSCs, such as ASCs) used in any one of the methods described herein contains around 10% DMSO and around 90% FCS (or FBS). For example, the cryopreservation medium for a population of MSCs, such as human ASCs may contain between 5-15% DMSO in FBS. The freezing medium for a population of human embryonic stem cells may contain 10% DMSO, 30% FBS and 60% conditioned HES medium.

DMSO may be used in combination with human serum albumin. For example, the cryopreservation medium may contain between about 2-10% human serum albumin and between about 5-15% DMSO. A particularly preferred cryopreservation medium contains around 10% DMSO and around 5% human serum albumin.

Other molecules such as glycerol, ethylene glycol, hydroxycellulose, or the disaccharides sucrose, maltose, and trehalose have been shown to enhance cell viability when combined with DMSO in a freezing medium.

Trehalose is a disaccharide found at high concentrations in a wide variety of organisms that are capable of surviving almost complete dehydration and has been shown to stabilize certain cells during freezing. Trehalose is thought to maintain thermodynamic stability of membranes by preserving phospholipid head group spacing and inhibiting lipid phase transitions and separation during freezing. Trehalose is that it does not easily penetrate lipid bilayers, and must be loaded into cells through endocytosis or other methods that temporarily disrupt the cell membrane. For example, the cryopreservation medium for ASCs may contain trehalose at a concentration of between about 50-200 mM, such as around 100 mM. Trehalose can be used to reduce the potential toxicity associated with other cryoprotectants, e.g. when used in combination with DMSO at the concentrations discussed above (see, e.g. Buchanan et al. Cell Preservation Technology (2005) 3(4): 212-222).

Polyvinylpyrrolidone (PVP), sericin and maltose, and methyl cellulose (MC) are alternative cryopreservation agents. These compounds have been tested as cryopreservation solutions, e.g. of ASCs, as alternatives to DMSO or to animal-derived serum (Miyagi-Shiohira et al. Cell Medicine (2015) 8: 3-7).

PVP, which is a macromolecular polymer, lowers the freezing point and inhibits the increase of extracellular salt concentration, thereby stabilizing the cell membrane during the freeze-thaw process. PVP can be added to the cryopreservation medium at levels of between about 1% and 40%, such as between about 8 and 25%, e.g. about 1%, 5%, 10%, 20% or 40%. The cryopreservation medium can also contain human serum, optionally between about 5-20% (e.g. 10% human serum) in addition to PVP. For example, the cryopreservation medium for ASCs may contain 10% PVP and 10% human serum.

MC is a macromolecular polymer that can substitute animal-derived serum in cryopreservation solutions, although the presence of DMSO (or another cryopreservation agent) is essential to retain cellular activity after the freeze-thaw process. A cryopreservation medium may contain between about 0.5% and 2% w/v MC, e.g. about 1% w/v, in combination with a suitable concentration of DMSO as discussed above. For example, the cryopreservation medium may contain about 1% MC and about 10% DMSO.

Sericin is a cocoon-derived protein, which can also substitute animal-derived serum in cryopreservation solutions. A cryopreservation medium may contain between about 0.5% and 2% w/v sericin, e.g. about 1% w/v. Sericin may be used in combination with maltose (e.g. 50-200 mM maltose) and/or a suitable concentration of DMSO as discussed above. For example, the cryopreservation medium may contain about 1% sericin, 100 mM maltose and 10% DMSO.

There are various commercially available cryopreservation solutions, for example: FM-1 (Kyokuto Pharmaceutical Industrial Co., Ltd, Tokyo, Japan), the cell banker cryoprotectant series (Nippon Zenyaku Kogyo Co., Ltd., Fukushima, Japan); CryoStor (Stem Cell Technologies); Synth-a-Freeze cryopreservation medium (Thermo Fisher Scientific) and MesenCult™-ACF Freezing Medium (Stem Cell Technologies).

The cell banker cryoprotectant series allows for rapid cell cryopreservation at −80° C. and its use is associated with improved survival rate following freezing and thawing. Serum-containing cell bankers 1 and 1+ can be used for cryopreservation of almost all mammalian cells. Moreover, non-serum-type cell banker 2 allows cryopreservation of cells in serum-free culture conditions. STEMCELLBANKER (cell banker 3), on the other hand, is a cell cryopreservation solution that is chemically defined, is xeno-free (i.e. contains no non-human animal products) and optimizes the preservation performance of stem cells, such as somatic and induced pluripotent stem cells.

The CryoStor® range (BioLife Solutions, Inc.) is a serum-free, animal component-free, and defined cryopreservation media containing various concentrations of DMSO (CS10 10% DMSO; CS5 5% DMSO; CS2 2% DMSO). CryoStor® CS10 is has been used for the cryopreservation of MSCs (including ASCs), embryonic stem (ES) and induced pluripotent stem cells (iPS). Synth-a-Freeze cryopreservation medium (Thermo Fisher Scientific) has been used to cryopreserve induced pluripotent stem cells (iPS).

Cell specific cryopreservation media are also available, such as mFreSR™ and FreSR™-S Cryopreservation Media for ES and iPS cells, MesenCult™-ACF Freezing Medium for MSCs, and STEMdiff™ Neural Progenitor Freezing Medium for neural progenitor cells derived from ES/iPS cells. For example, MSCs can be cryopreserved in MesenCult™-ACF Freezing Medium (Stem Cell Technologies), which can be used following MSC culture in MesenCult™-ACF Plus or MesenCult™ media (Stem Cell Technologies) to cryopreserve MSCs

Exemplary cryopreservation media and cryoprotectants used for various stem cell types are shown in the table below:

| | Freezing | Cryopreservation medium or | |
|---|---|---|---|
| Stem cell type | protocol | cryoprotectant used | Reference |
| Human | Vitrification | 20% DMSO, 20% ethylene glycol | Li et al. Fertil Steril. (2010) |
| embryonic stem | | (EG) and 0.5 mol/L sucrose (after | 93(3): 999 |
| cells | | equilibration at a lower | |
| | | concentration of DMSO and EG) | |
| | Slow freezing | 5% DMSO, 10% EG and 50% FBS | Ha et al. Hum. Reprod. (2005) |
| | to −80° C. at | | 20: 1779-85 |
| | 1° C./min | | |
| Mesenchymal | Slow freezing | Culture media supplemented with | Carvalho et al. Transplant |
| stem cells (bone | to −80° C. at | 10% FCS and 5% DMSO | Proc. (2008) 40: 839-41 |
| marrow derived) | 1° C./min | | |
| | Slow freezing | Parental solutions (e.g. saline, | Pal et al. J Tissue Eng. Regen. |
| | to −80° C. at | Plasmalyte A) supplemented with | Med. (2008) 2: 436-44 |
| | 1° C./min | 5% HSA and 10% DMSO | |
| | Slow freezing | 5% DMSO | Haack-sørensen et al. Methods |
| | to −80° C. at | | in Molecular Biology, Humana |
| | 1° C./min | | Press, Totowa, NJ, 2011, pp. |
| | | | 161-174. |
| | Slow freezing | 10% DMSO | Xu et al. J. Tissue Eng. Regen. |
| | to −80° C. at | | Med. 8 (2014) 664-672. |
| | 1° C./min | | |
| | 4° C. for 10 | CellBanker (commercial DMSO- | Kotobuki et al. Tissue Eng. 11 |
| | min; −30° C. for | based) | (2005) 663-673. |
| | 1 h; −80° C. for | | |
| | 2-3 h | | |
| Mesenchymal | Vitrification | 40% EG, 18% Ficoll 70 and 0.3M | Moon et al. Hum |
| stem cells | | sucrose | Reprod.(2008) 23: 1760-70 |
| (human amnion | Uncontrolled | DMSO or glycerol (5 or 10%); | Janz et al. J. Biomed. |
| derived) | (−20° C. for | sucrose (30 or 60 mM); trehalose | Biotechnol. (2012) 649353. |
| | 20 min; −80° C. | (60 or 100 mM) | |
| | for 12-16 h) or | | |
| | controlled (1° | | |
| | C./min to −60° | | |
| | C.; 3° C./min | | |
| | to −100° C.) | | |
| Mesenchymal | Vitrification | 20% DMSO, 20% EG, 0.5M | Todorov et al. Cell Biol. Int. 34 |
| stem cells | | sucrose | (2010) 455-462. |
| (human foetal | | | |
| liver) | | | |
| Mesenchymal | Slow freezing | 10% DMSO, 90% FBS | Barcia et al. Cytotherapy, |
| stem cells | to −150° C. at | | (2017); 19(3): 360-370 |
| (umbilical cord | 1° C./min | | |
| tissue-derived) | | | |
| Mesenchymal | Slow freezing | 10% DMSO | Liu et al. Cryobiology (2008) |
| stem cells | | | 57(1): 18-24 |
| (adipose derived) | Slow freezing | 80% FCS or human serum and 10% | Thirumala et al. Stem Cells |
| | | DMSO | Dev. (2010) 19(4): 513-522 |
| | Slow freezing | 10% PVP and 10% human serum | |
| | Slow freezing | 1% methyl cellulose and 10% | |
| | | DMSO | |
| | Slow freezing | 10% DMSO, 1% sericin and 0.1 | Miyamoto et al. Cell |
| | | mol/L maltose | Transplant. (2012) 21(2-3): |
| | | | 617-622 |
| | −20° C. for 30 | 4% DMSO, 6% trehalose | De Rosa et al. Tissue Eng. Part |
| | min; −80° C. for | | C Methods 15 (2009) 660-667. |
| | 1 h | | |
| Mesenchymal | 4° C. for 1 h; | 10% DMSO or 10% glycerol or | Ding et al. J. Cell. Physiol. 223 |
| stem cells | −20° C. for 2 h; | 10% EG | (2010) 415-422. |
| (human teeth) | −80° C. | | |
| | overnight | | |
| Mesenchymal | ~1° C./min in | 0.5/1/1.5M EG or propylene glycol | Woodset al. Cryobiology 59 |
| stem cells | freezing | or DMSO | (2009) 150-157. |
| (human dental | container at | | |
| pulp) | −85° C. for 24 h | | |
| Haematopoietic | Slow freezing | 10% DMSO | Berz et al. Am J Hematol. |
| stem cells | to −80° C. at | | (2007) 82: 463-72 |
| | 1° C./min | | |

Further details regarding the cryopreservation of MSCs is provided in, for example, Marquez-Curtis et al. (Cryobiology (2015) 71(2): 181-197) and Francois et al. (Cytotherapy (2012) 14(2): 147-152).

### Freezing Protocol and Storage Conditions

The freezing rate must be fast enough to avoid solute and electrolyte imbalances that cause cell dehydration and damage, and slow enough to prevent extracellular and intracellular ice crystal formation. Cryoprotectants reduce the freezing point of the medium, so the mixture of cells, and cryopreservation medium containing a cryoprotectant, is a eutectic system because the combined freezing point is lower than the individual components. During the freezing process, fluids move from lower solute concentrations in unfrozen cells into partially frozen medium while the plasma membrane prevents entrance of extracellular ice crystals. Slow freezing permits fluids to move out of the cells at a rate that results in balanced osmotic pressure between cell and medium by the time the medium freezes. If the rate is too slow, cells are fatally dehydrated or their plasma membranes are irreversibly damaged. If the rate is too high, there is insufficient fluid migration and the cells retain high levels of freezable water during the cryopreservation process, which results in lethal intracellular ice damage.

A mechanical or a controlled rate freezer may be used to freeze the population of stem cells in the methods described herein. A controlled rate freezer can be programmed to cool the cells to around −80° C. at a particular rate. A typical freezing rate for cryopreservation of most cells (including MSCs) to −80° C. is −1° C./minute. Such as freezing rate can be achieved by insulating the population of stem cells before placing them in a mechanical −80° C. freezer, using for example a closed-cell polyethylene foam container (e.g. CoolCell®; BioCision), a styrofoam container or an isopropanol (IPA)-filled container (e.g. Mr. Frosty™ (Thermo Scientific)). CoolCell® and Mr. Frosty™ both have a stated freeze rate of −1° C./minute. The freezing protocol may require optimisation for a given cell type or line, however, to achieve maximum viability and maintenance of function upon thaw. In the methods described herein, the freezing step(s) may be carried out at a rate in the range of about −0.5 to about −10° C./minute, preferably about −3 to about −5° C./minute, e.g. around −1, −2, −3, −4, −5 or −10° C./minute. The final freezing temperature may be between about −70° C. to about −130° C., Thus, in the disclosed methods the freezing step(s) may comprise reducing the temperature to between −70° C. and −130° C. at a rate of between about −0.5 to about −10° C./minute. The temperature may be reduced from +4° C. to between −100-180° C. in 10-60 mins. The population of stem cells can be frozen at any cell density. A preferred cell density of the frozen population of stem cells is in the range of around 1 to around 50 million cells/mL, preferably around 25 million cells/mL.

After freezing, the frozen population of cells may be stored in liquid nitrogen at −196° C. until required. Thermally dependent metabolic processes do not typically occur below −100° C., so stem cells are in metabolic stasis in liquid nitrogen. For temperatures above −100° C. where low-temperature mechanical stresses are less severe, a variety of containers may be used. However, when storing material at liquid nitrogen temperatures, containers specifically designed to withstand cryogenic temperatures (i.e. “cryovials”) must be used. A variety of containers specifically designed for cryogenic use are commercially available, including plastic cryovials (e.g. with screw top closures) or glass ampoules (which may be flame sealed). Commonly used sizes are 1.2, 2.0, 4, 5, 10 and 15 mL cryovials (see, e.g. Nalgene® and TruCool® vials). Generally, 0.5-1.0 mL of the cell suspension is placed into a 1.2 or 2.0 mL vial. Various sizes and types of liquid nitrogen storage container are commercially available (see e.g. the Thermo Scientific™ Locator™ Plus systems and CryoExtra™ High-Efficiency cryogenic storage systems).

In a preferred embodiment, the population of cells (e.g. ASCs) are frozen in a cryopreservation medium (e.g. 10% DMSO in FBS) in one or more cryovial at −80° C. and then transferred to a liquid nitrogen storage container.

The methods of stem cell cryopreservation described herein may comprise freezing a population of stem cells, such as ASCs, in a plurality of cryovials. The population of stem cells in each of the plurality of cryovials may be identical, i.e. aliquots of a single population of stem cells obtained from any one of the methods disclosed herein. In some instances, the method may further comprise repeating the steps of any one of the methods of stem cell cryopreservation described herein for a plurality of populations of stem cells. The repeated steps may be carried out in series, i.e. following on from the previous method steps. Alternatively, the repeated steps may be carried out in parallel, i.e. the method steps are carried out for the plurality of populations of stem cells at the same time. Each repeat may comprise the same method steps, or may comprise different method steps as described herein. The plurality of populations of stem cells may comprise populations of stem cells (e.g. ASCs) obtained from the same donor (e.g. where different populations are obtained by using the same method steps described herein in a separate procedure(s), or by using a different method(s) as described herein). The plurality of populations of stem cells may be populations of stem cells (e.g. ASCs) obtained from different donors. Alternatively, the plurality of populations of stem cells may comprise different types of MSCs. For example, the plurality of populations of stem cells may comprise one or more, two or more, three or more of the following MSCs: MSCs derived from bone marrow, umbilical cord, dental pulp, blood (e.g. peripheral, cord or menstrual), placenta and adipose. These methods may also comprise freezing the plurality of populations of stem cells in a plurality of cryovials. The methods may further comprise storing the plurality of cryovials in a liquid nitrogen storage container for at least one month, at least 2 months, at least 3 months, at least 6 months, or at least 1 year. The cryovials may be frozen at −80° C. and then transferred to a liquid nitrogen storage container. A plurality of cryovials is more than one cryovial, e.g. at least about 10, at least about 20, at least about 50, about 100, about 1000, about 2000 or about 5000 or more cryovials.

Also provided herein is a liquid nitrogen storage container containing the plurality of cryopreservation vials obtained according to the methods described herein.

Vitrification is another form of cooling that involves extremely rapid (>−1000° C./second) cooling of cells immersed in a cryopreservation medium within open storage vessel. Rapid freezing can be achieved by plunging the sample in a cryovial into liquid nitrogen. This process inhibits ice formation, although it requires potentially cytotoxic concentrations of cryoprotectants and the use of an open container risks contamination. Vitrification has been successfully to cryopreserve human embyronic stem cells (hESCs). Capillary vitrification of human embryonic stem cells in cryopreservation media containing DMSO and ethylene glycol has been shown to enhance survival of cryopreserved cells greater than an order of magnitude as compared to slow freezing and fast thawing methods. Briefly, colonies of hEScs (100-400 cells) are placed in a cryopreservation medium comprising 20% DMSO, 20% ethylene glycol and 0.5 M sucrose, after equilibration in a lower DMSO and EG solution. The colonies are loaded into straws and plunged into liquid nitrogen.

### Thawing Protocol

Typically, cells are thawed at or near their growth temperature, e.g. ˜37° C. Thus, in the methods disclosed herein, the population of stem cells may be thawed at 37° C.

Cells pass through a temperature for ice crystal formation, −15° C. to −60° C., during freezing and thawing. Rapid thawing at around 90-100° C./minute by immersion in a 37° C. waterbath is often employed to prevent ice crystal formation. However, thawing at a lower temperature or slower rate may reduce certain types of damage, such as oxidative stress detected by adhesion-mediated signaling, while permitting membranes to seal any pores formed by ice crystallisation. In the methods described herein, the population of stem cells is typically thawed at 37° C. This rapid thaw step can be achieved by plunging the cells in a cryovial into a waterbath at 37° C. The thawing protocol may require optimisation for a given cell type or line, however, to achieve maximum viability and/or maintenance of cell function.

The thawed cells can be washed to remove the cryopreservation medium, prior to culture. The examples of washing methods discussed above (e.g. in relation to removal of NAC and/or media exchange) are suitable for this purpose also.

### Post-Thaw Assessment

Post thaw assessment of the population of stem cells (e.g. to check on the impact of NAC pretreatment or post-thaw treatment) may include one or more (or all) of the following tests: cell viability, morphology, cell surface marker assessment, differentiation assays and analysis of other functional properties. Exemplary assessments are provided in the examples.

### Viability

As used herein the term “viability” or “viable” refers to a cell that is capable of normal growth and development after having been cryopreserved and thawed. Thus, assessing the viability of the population of stem cells relative to a similar population of stem cells that has not been subjected to pretreatment with NAC, post-thaw treatment with NAC, or both, can be used to confirm that the cells are not negatively affected (i.e. decreased viability) as a result of pretreatment and/or post-thaw treatment with NAC (pretreatment and/or post-thaw treatment with NAC may, however, have a positive effect on viable cell number, growth rate and recover rate etc, as discussed further below).

Examples of experiments that can be used in the disclosed methods to determine the level of cell viability include trypan blue staining and MTS assays, as discussed in the examples. The MTS assay is a measure of functional viability (i.e. metabolism), while the trypan blue assay measure structural viability (i.e. membrane integrity). Other methods known to those skilled in the art, such as alamar blue assays, may also be used for cell viability measurements.

MTS assay is a colorimetric method for determining the number of viable cells in proliferation or cytotoxicity assays. For example, the CellTiter 96® AQueous One Solution Reagent contains a novel tetrazolium compound [3-(4,5-dimethylthiazol-2-yl)-5-(3-carboxymethoxyphenyl)-2-(4-sulfophenyl)-2H-tetrazolium, inner salt; MTS(a)] and an electron coupling reagent (phenazine ethosulfate; PES). PES has enhanced chemical stability, which allows it to be combined with MTS to form a stable solution. This convenient “One Solution” format is an improvement over the first version of the CellTiter 96® AQueous Assay, where phenazine methosulfate (PMS) is used as the electron coupling reagent, and the PMS Solution and MTS Solution are supplied separately. The MTS tetrazolium compound (Owen's reagent) is bioreduced by metabolically active cells into a coloured formazan product that is soluble in tissue culture medium. Assays can be performed by adding a small amount of the CellTiter 96® AQueous One Solution Reagent directly to culture wells, incubating for 1-4 hours and then recording the absorbance at 490 nm with a 96-well plate reader.

### Differentiation Capacity

Following cryopreservation, for stems cells to be applicable for a variety of therapeutic applications, the cells must remain viable, be maintained in an undifferentiated state and retain their differentiation capacity. Any differentiation will limit their use in downstream applications. Thus, assessing the differentiation capacity of the population of stem cells relative to a similar population of stem cells that has not been subjected to pretreatment with NAC, post-thaw treatment with NAC, or both, can be used to confirm that the identity of the cells is unaffected by pretreatment and/or post-thaw treatment with NAC.

Herein the term “differentiation” or “differentiate’ refers to a process during which pluripotent or multipotent (unspecialized) stem cells change into a more specialized cell type.

One way to determine differentiation potential or pluripotency in embryonic or induced pluripotent stem cells is to measure the level of surface markers such as OCT4 and SSEA-4, e.g. by immunofluorescence microscopy (Xu, C., et al., (2001) Nat Biotechnol. 19: 971-974). OCT4 and SSEA-4 are markers of undifferentiated stem cells (i.e. that have the potential to differentiate to other lineages). OCT4 is an embryonic gene transcription factor that plays a role in control of developmental pluripotency, so that when OCT4 gene activity is repressed in pluripotent stem cells differentiation, differentiation occurs. SSEA4 expression can also be determined by flow cytometry.

MSCs have the ability to differentiate into different tissues, such as bone, cartilage, tendon and fat tissue. They are considered multipotent adult progenitor cells, because their differentiation potential is more restricted than that of pluripotent/totipotent stem cells, such as embryonic or induced pluripotent stem cells, that have the potential to differentiate into all adult tissues (Jiang et al., (2002) Nature 418(6893):41-49). Methods of testing the differential potential of MSCs in different tissues are known in the prior art (e.g. Guilak et al., J Cell Physiol. (2006) 206(1): 229-237; Zuk et al., Mol Biol Cell. (2002) 13(12): 4279-4295).

Cell Morphology and/or Size

The phenotype of the population of stem cells may be assessed by morphology and/or size. The term “phenotype” refers to the observable characteristics of a cell, such as size, morphology, protein expression, including of cell surface markers etc. Thus, assessing the cell morphology and/or size of the population of stem cells relative to a similar population of stem cells that has not been subjected to pretreatment with NAC, post-thaw treatment with NAC, or both, can be used to confirm that the identity of the cells is unaffected by pretreatment and/or post-thaw treatment with NAC.

Cell morphology and/or size can be viewed and imaged using an inverted culture microscope.

Human iPSC and ESC share similar characteristics, including morphology, proliferation, surface markers, gene expression, in vitro differentiation capability and teratoma formation (see, e.g. Thomson et al. Science (1998) 282(5391): 1145-1147; Xu et al. Nat. Biotechnol. (2001) 19(10): 971-974; Takahashi et al. Cell (2007) 131(5): 861-872; Courtot et al. Biores. Open Access (2014) 3(5): 206-216; Kato et al. Scientific Reports (2016) 6: 34009).

Depending on the tissue of origin, MSCs are morphologically and immunophenotypically similar but not identical (Colter et al., Proc. Natl Acad. Sci. USA (2000) 97(7): 3213-3218; Kern et al., Stem Cells (2006) 24(5): 1294-1301; Huang et al., J. Dent. Res. (2009) 88(9): 792-806; Carvalho et al., Curr. Stem Cell Res. Ther. (2011) 6(3): 221-8; Harris et al., Curr. Stem Cell Res. Ther. (2013) 8(5): 394-9; Li et al., Ann N Y Acad Sci. (2016) 1370(1): 109-118).

### Characterisation of Cell Surface Markers

The phenotypic characterization of a stem cell population can be carried out by analysing one or more cell surface markers. Thus, assessing the expression of one or more cell surface markers on the population of stem cells relative to a similar population of stem cells that has not been subjected to pretreatment with NAC, post-thaw treatment with NAC, or both, can be used to confirm that the identity of the cells is unaffected by pretreatment and/or post-thaw treatment with NAC The presence or absence of antibody binding to a cell surface marker of interest may be determined by different methods that include but are not limited to immunofluorescence microscopy, radiography and flow cytometry. The determination of the profile of expression of surface markers by antibodies may be direct, using a labelled antibody, or it can be indirect, using a second labelled antibody against a primary specific antibody to the cell marker of interest, thus achieving signal amplification. In flow cytometry, by using a labelled antibody the level of fluorochrome can be correlated with the quantity cell surface marker bound specifically to the antibody. The differential expression of one or more cell surface markers in a stem cell population allows the identification and/or isolation of said population, e.g. using FACS (Fluorescence Activated Cell Sorting).

For example, according to the International Society for Cellular Therapy, the minimal criteria to define MSCs may be the expression of CD105, CD73, CD44 and CD90, and lack of expression of CD45, CD14 or CD11b, CD79alpha or CD19 and HLA class II (Dominici et al., Cytotherapy. (2006) 8(4): 315-7). Examples of antibodies that can used to assess the CD73, CD90 and CD105 markers are provided in Example 5. Antibodies that can be used to assess the other markers are commercially available e.g. from Beckton Dickinson, examples of which are listed below.

| Marker | Fluorochrome | Antibody source |
|---|---|---|
| CD45 | FITC | Mouse IgG1k |
| CD34 | APC | Mouse IgG1 |
| CD14 | APC | Mouse IgG2ak |
| CD11b | PE | Mouse IgG1k |
| CD79alpha | PE | Mouse IgG1k |
| CD19 | APC | Mouse IgG1 |
| HLA class II | APC | Mouse IgG1 |

For example, post-thaw assessment of a population of ASCs can be carried out by checking for expression of CD29, CD73, CD90 and CD105 (e.g. as in Example 5). Such an analysis can be used to confirm that the identity of the cells is unaffected by pretreatment or post-thaw treatment with NAC.

Cell surface markers associated with a particular stem cell type are known and are exemplified below.

### Other Functional Properties

Assessing other functional properties of the population of stem cells (relative to a similar population of stem cells that has not been subjected to pretreatment with NAC, post-thaw treatment with NAC, or both) can be used to confirm that the identity of the cells is unaffected by pretreatment and/or post-thaw treatment with NAC. For example, for ASCs, other functional properties that can be assessed include: the capacity of ASCs to inhibit the proliferation of stimulated lymphocytes (e.g. as in Example 6); the immunomodulatory capacity of ASCs, e.g. on monocyte differentiation (e.g. as in Example 7); the capacity of ASCs to modulate phagocytosis, e.g. of Staphylococcus aureus particles, by mature dendritic cells (mDCs); the ASC-mediated upregulation of one or both of CD206 and CD163 on the cell surface of mDCs (e.g. as in Example 9); and/or the ASC-mediated modulation of CD14-CD1a+ mDCs to CD14+CD1a− mDCs (e.g. as in Example 9).

Thus, in any of the methods disclosed herein, the thawed population of ASCs can be assessed for one or more, two or more, three or more, four or more, five or more, six or more or all seven of the following properties: (1) cell viability; (2) expression of cell surface markers CD29, CD73, CD90 and CD105; (3) capacity to inhibit the proliferation of stimulated lymphocytes; (4) immunomodulatory effect on monocyte differentiation; (5) capacity to modulate phagocytosis by mature dendritic cells, e.g. of Staphylococcus aureus particles; (6) capacity to upregulate of one or both of CD206 and CD163 on the cell surface of mDCs; and (7) modulation of CD14-CD1a+ mDCs to CD14+CD1a− mDCs, For each properties, the assessment can be performed relative to a similar population of ASCs that has not been subjected to pretreatment with NAC, post-thaw treatment with NAC, or both, allowing confirmation that the identity of the cells is unaffected and/or that cell viability is not negatively affected (i.e. decreased cell viability) by pretreatment and/or post-thaw treatment with NAC. Similarly, also disclosed is a population of ASCs obtained by any one of the methods described herein that possesses one or more, two or more, three or more, four or more, five or more, six or more or all seven of these properties (e.g. as assessed relative to a similar population of ASCs that has not been subjected to pretreatment with NAC, post-thaw treatment with NAC, or both, as discussed above).

### Types of Stem Cells

The population of stem cells may be a population of pluripotent stem cells or a population of mesenchymal stem cells (MSCs), e.g. bone-marrow derived, umbilical cord tissue-derived, blood-derived (including cord blood derived), menstrual, dental pulp-derived, placental-derived or adipose-derived MSCs (Huang et al., J. Dent. Res. (2009) 88(9): 792-806; Carvalho et al., Curr. Stem Cell Res. Ther. (2011) 6(3): 221-8; Harris et al., Curr Stem Cell Res Ther. (2013) 8(5): 394-9; Li et al., Ann. N Y Acad. Sci. (2016) 1370(1): 109-18). In a preferred embodiment, the stem cells are human cells (e.g. human ASCs). In preferred embodiments of the invention, the population of stem cells are adipose-derived stromal stem cells (ASCs). The ASCs used in the methods of cryopreservation described herein may be an expanded population of ASCs.

Methods for producing and culturing populations of stem cells according to the invention are well known.

The population of stem cells may be substantially pure. The term “substantially pure” in relation to a population of stem cells (e.g. a MSC population such as a population of ASCs) refers to a stem cell population that is least about 75%, typically at least about 85%, more typically at least about 90%, and most typically at least about 95% homogenous. Homogeneity can be assessed by morphology and/or by cell surface marker profile. Techniques for assessing morphology and cell surface marker profile are disclosed herein.

### Pluripotent Stem Cells

There are two sources of pluripotent stem cells. First, embryonic stem cells (ESCs) are derived from the inner cell mass of a pre-implantation blastocyst and pluripotency is controlled by an intrinsic regulatory network of core transcription factors, octamer-binding transcription factor 4 (OCT4), sex determining region Y-box 2 (SOX2), and Nanog homeobox (NANOG). In one embodiment, an embryonic stem cell line is used. An embryonic stem cell line comprises constantly dividing cells produced from a group of parent cells which were harvested from a single embryo. The embryonic stem cell line used in the present invention is not obtained by destruction of a human embryo. Embryonic stem cell lines are commercially available, for example from ATCC. The embryonic stem cells of the embryonic stem cell line do not lose their pluripotency while they are in culture. In particular, the embryonic stem cells of the embryonic stem cell line do not differentiate while they are in culture. Second, induced pluripotent stem cells (iPSCs) are derived by the ectopic or elevated expression of four transcription factors, OCT4, SOX2, Kruppel like factor 4 (KLF4), and MYC proto-oncogene (C-MYC) essential for induction of pluripotency in somatic cells.

Techniques for isolating stable (undifferentiated) cultures of embryonic stem cells, such as human embryonic stem cells, are well established (e.g. U.S. Pat. No. 5,843,780; Thomson et al., Science (1998) 282: 1145-1147; Turksen & Troy (2006) Human Embryonic Stem Cells. In: Turksen K. (eds) Human Embryonic Stem Cell Protocols. Methods in Molecular Biology, volume 331, Humana Press; Sevilla et al., Stem Cell Research (2017) 25: 217-220; and Mitalipova & Palmarini (2006) Isolation and Characterization of Human Embryonic Stem Cells. In: Turksen K. (eds) Human Embryonic Stem Cell Protocols. Methods in Molecular Biology, volume 331, Humana Press). In one embodiment, the method for obtaining embryonic stem cells does not include the destruction of one or more human embryos.

Techniques for producing iPSCs are well established since their discovery in 2007 by Yamanaka's group (e.g. Takahashi et al., Cell (2007) 131(5): 861-72). Since then, new improved methods for iPSC generation have been developed, including non-integration and feeder free methodologies and automated high-throughput derivation (Paull et al., Nature Methods (2015) 12(9): 885-892).

iPSC are characterized by the expression of a battery of pluripotency markers: NANOG, SOX2, SSEA4, TRA1-81, TRA1-60, and the lack of lineage-specific markers. The pluripotency of iPSC is demonstrated by their capacity to differentiate into the three germ layers in the embryoid body assay, with posterior analysis of differentiation markers from the three germ layers Tuj1 (ectoderm marker), SMA (mesoderm marker) and SOX17 (endoderm marker) by immunohistochemistry (Paull et al., Nature Methods (2015) 12(9): 885-892.

### MSCs

“Mesenchymal stem cells” (also referred to herein as “MSCs”) are multipotent stromal cells. They are typical derived from connective tissue, and are non-hematopoietic cells. The population of MSCs (according to Dominici et al. 2006 (Cytotherapy 8(4): 315-317), may: (1) adhere to plastic under standard culture conditions (e.g. a minimal essential medium plus 20% fetal bovine serum); (2) express (i.e. greater than or equal to 80% of population of MSCs) CD105, CD90, CD73 and CD44; (3) lack expression (e.g. less than or equal to 5% of the MSC population) of CD45, CD14 or CD11b, CD79a or CD19, and HLA-DR (HLA Class II); (4) be capable of differentiating into osteoblasts, adipocytes and chondroblasts.

MSCs can be obtained using standard methods from, for example, bone marrow, umbilical cord tissue and blood, menstrual, dental pulp, cord blood, placental and adipose tissues.

Although MSCs obtained from different tissues are similar, they have some differences in phenotypical and functional characteristics. For example, the expression levels of cell surface markers CD54 and CD106 may differ depending on the source/origin of the MSCs. These can be measured by flow cytometry. The mRNA levels of some genes such as SOX2, IL1alpha, IL1beta, IL6 and IL8, may be differentially expressed by MSCs from different tissues, and can be measured by routine methods. IL6 and PGE2 secretion may also be different between MSC from different origins, and thus the cells may have different modulatory capacity (see, e.g. Yang et al. PLoS ONE (2013) 8(3) e59354).

### Bone Marrow Derived MSCs (BMSCs)

Bone-marrow mesenchymal stem cells (BM-MSCs) are similar to MSCs from other tissue sources. However, they have some differences in phenotypical and functional characteristics compared to MSCs from other tissue origins, such as umbilical-cord MSCs, placental MSCs, dental pulp MSCs, and menstrual MSCs. Even though their minimal characterization criteria is common, including their capacity to adhere to plastic, minimal surface identity markers and capacity to differentiate into bone, cartilage, tendon and fatty tissue, they all have some slight differences. These peculiarities include different expression levels of some surface markers, such as CD105, different levels of secreted soluble factors implicated in their immunomodulatory potential and regenerative potential, and in general, slightly different functional properties that may make each source or origin more suitable for specific therapeutic indications (Miura et al., Int J Hematology (2016) 103(2): 122-128; Wuchter et al., Cytotherapy (2015) 17(2): 128-139; Wright et al., Stem Cells (2011) 29(2): 169-178).

### Umbilical Cord Derived and Dental Pulp Derived MSCs

Huang et al. (J. Dent. Res. (2009) 88(9): 792-806) discusses MSCs from dental pulp and compares their characteristics with MSCs from other sources. Carvalho et al. (Curr Stem Cell Res Ther. (2011) 6(3): 221-228) and Harris et al. (Curr Stem Cell Res Ther. (2013) 8(5): 394-399) discuss umbilical cord-derived MSCs, their characterisation (including phenotype and secretome) and applications thereof.

### ASCs

Adipose-derived MSCs (ASCs) are normally isolated from subcutaneous adipose tissue, which allows them to be acquired in large numbers. ASCs proliferate rapidly with a high cellular activity, making them an ideal source for obtaining MSCs.

By “adipose tissue” is meant any fat tissue. The adipose tissue may be brown or white adipose tissue, derived from subcutaneous, omental/visceral, mammary, gonadal, or other adipose tissue site. Typically, the adipose tissue is subcutaneous white adipose tissue. Such cells may comprise a primary cell culture or an immortalized cell line. The adipose tissue may be from any organism having fat tissue. Typically, the adipose tissue is mammalian, most typically the adipose tissue is human. A convenient source of adipose tissue is from liposuction surgery, however, the source of adipose tissue or the method of isolation of adipose tissue is not critical to the invention.

The population of stem cells may be a population of ASCs produced using the methods described in Example 1, or any one of the methods described herein.

The preferred ASCs are the human allogeneic adipose-derived stem cells (human eASCs) authorised in the product “Darvadstrocel” (tradename “Alofisel®). These expanded ASCs express the cell surface markers CD29, CD73, CD90 and CD105. The cells are capable of expressing factors such as vascular endothelial growth factor (VEGF), transforming growth factor-beta 1 (TGF-β1), interleukin 6 (IL-6), matrix metalloproteinase inhibitor-1 (TIMP-1) and interferon-gamma (IFN-γ) and inducible indoleamine 2,3-dioxygenase (IDO). Thus, the population of ASCs may be characterised in that at least about 50%, at least about 60%; at least about 70%; at least about 80%; at least about 85%; at least about 90% or at least about 95% or more express one or more of CD29, CD73, CD90 and/or CD105. The population of ASCs may be characterised in that at least about 50%, at least about 60%; at least about 70%; at least about 80%; at least about 85%; at least about 90% or at least about 95% of the population of cells express all of CD29, CD73, CD90 and CD105. Typically, the population of ASCs may be characterised in that at least about 80% of the population of cells express all of CD29, CD73, CD90 and CD105.

According to Bourin et al. (Cytotherapy (2013) 15(6): 641-648), a population of ASCs may be defined as being positive for expression of CD13, CD29, CD44, CD73, CD90 and CD105, and negative for expression of CD31 and CD45. In the population of ASCs, at least about 50%, at least about 60%; at least about 70%; at least about 80%; at least about 85%; at least about 90% or at least about 95% of the population of cells may express CD13, CD29, CD44, CD73, CD90 and CD105, and fewer than about 5%, about 4%, about 3% or about 2% of the population of ASCs may express CD31 and CD45. Typically, in the population of ASCs, at least about 80% of the population of cells may express CD13, CD29, CD44, CD73, CD90 and CD105, and fewer than about 5% of the population of ASCs may express CD31 and CD45.

The ASCs may be adherent to plastic under standard culture conditions.

Expanded ASC (eASC) exhibit a fibroblast-like morphology in culture. Specifically, these cells are big and are morphologically characterised by a shallow cell body with few cell projections that are long and thin. The nucleus is large and round with a prominent nucleolus, giving the nucleus a clear appearance. Most of eASCS display this spindle-shaped morphology, but it is usual that some of the cells acquire polygonal morphologies (Zuk et al. Tissue Eng (2001) 7(2): 211-228).

The ASCs may be positive for the surface markers HLA I, CD29, CD44, CD59, CD73, CD90, and CD105. In some embodiments, the population of ASCs may be characterised in that at least about 50%, at least about 60%, at least about 70%, at least about 80%, at least about 85%; at least about 90% or at least about 95% of the population of ASCs express the surface markers HLA I, CD29, CD44, CD59, CD73, CD90, and CD105. Typically, at least about 80% of the eASCs express the surface markers HLA I, CD29, CD44, CD59, CD73, CD90, and CD105.

The ASCs may be negative for the surface markers HLAII, CD11b, CD11c, CD14, CD45, CD31, CD80 and CD86. In some embodiments, the population of ASCs may be characterised in that fewer than about 5% of the population of ASCs express the surface markers HLAII, CD11b, CD11c, CD14, CD45, CD31, CD80 and CD86. More typically, fewer than about 4%, 3% or 2% of the population of ASCs express the surface markers HLAII, CD11b, CD11c, CD14, CD45, CD31, CD80 and CD86. In one embodiment, fewer than about 1% of the population of ASCs express the surface markers HLAII, CD11b, CD11c, CD14, CD45, CD31, CD80 and CD86.

In some cases, in a population of ASCs at least about 80% of the population of cells express all of CD29, CD73, CD90 and CD105 and fewer than about 5% of the population of ASCs express the surface markers HLAII, CD11b, CD11c, CD14, CD45, CD31, CD80 and CD86.

In some embodiments the population of ASCs may express one or more (e.g. two or more, three or more, four or more, five or more, six or seven) of HLA I, CD29, CD44, CD59, CD73, CD90, and CD105. In some embodiments, the eASCs may not express one or more (e.g. two or more, three or more, four or more, five or more, six or more, seven or eight) of HLAII, CD11b, CD11c, CD14, CD45, CD31, CD80. In some embodiments, the eASCs express four or more of HLA I, CD29, CD44, CD59, CD73, CD90, and CD105 and do not express four or more of HLAII, CD11b, CD11c, CD14, CD45, CD31, CD80.

Expression of CD34 may be negative or low, e.g. expressed by 0 to about 30% of the population of ASCs. Thus, in some cases, the ASCs as described above may express CD34 at low levels, e.g. in about 5 to about 30% of the population. Alternatively, in other cases, the ASCs as described do not express CD34, e.g. fewer than about 5% of the population of ASCs express CD34.

In some embodiments, the population of ASCs (e.g. at least about 50%, at least about 60%, at least about 70%, at least about 80%, at least about 85%; at least about 90% or at least about 95% of the population of cells) may express one or more (e.g. two or more, three or more, four or more, five or more, six or more, seven or more, eight or more, nine or more, or ten or more (e.g. up to 13)) of the markers CD9, CD10, CD13, CD29, CD44, CD49A, CD51, CD54, CD55, CD58, CD59, CD90 and CD105. For example, the ASCs may express one or more (e.g. two, three or all) of the markers CD29, CD59, CD90 and CD105, e.g. CD59 and/or CD90.

In some embodiments the population of ASCs may not express one or more (e.g. two or more, three or more, four or more, five or more, six or more, seven or more, eight or more, nine or more, or ten or more (e.g. up to 15)) of the markers Factor VIII, alpha-actin, desmin, S-100, keratin, CD11b, CD11c, CD14, CD45, HLAII, CD31, CD45, STRO-1 and CD133, e.g. the ASCs do not express one or more (e.g. two, three or all) of the markers CD45, CD31 and CD14, e.g. CD31 and/or CD45.

In certain embodiments, the ASCs as described above (i) do not express markers specific for antigen presenting cells (APCs); (ii) do not express IDO constitutively; and/or (iii) do not significantly express MHC II constitutively. Typically expression of IDO or MEW II may be induced by stimulation with IFN-γ.

In certain embodiments, the ASCs as described above do not express Oct4.

### Methods of Preparing Populations of ASCs

Methods for the isolation and culture of ASCs to provide eASCs and population of stem cells of the invention, and compositions comprising populations of stem cells populations of the invention are known in the art. ASCs are typically prepared from the stromal fraction of adipose tissue and are selected by adherence to a suitable surface e.g. plastic. Thus, the methods of stem cell cryopreservation disclosed herein may comprise an initial step (prior to step (a) of any one of the methods) of: (i) isolating a population of ASCs from the stromal fraction of adipose tissue obtained from a patient, and (ii) culturing the population of ASCs. The ASCs can optionally be selected at step (i) for adherence to a suitable surface e.g. plastic. Optionally the phenotype of the ASCs may be assessed during and/or subsequent to the culturing step (ii).

ASCs can be obtained by any means standard in the art. Typically said cells are obtained disassociating the cells from the source tissue (e.g. lipoaspirate or adipose tissue), typically by treating the tissue with a digestive enzyme such as collagenase. The digested tissue matter is then typically filtered through a filter of between about 20 microns to 1 mm. The cells are then isolated (typically by centrifugation) and cultured on an adherent surface (typically tissue culture plates or flasks). Such methods are known in the art and e.g. as disclosed in U.S. Pat. No. 6,777,231. According to this methodology, lipoaspirates are obtained from adipose tissue and the cells derived therefrom. In the course of this methodology, the cells may be washed to remove contaminating debris and red blood cells, preferably with PBS. The cells are digested with collagenase (e.g. at 37° C. for 30 minutes, 0.075% collagenase; Type I, Invitrogen, Carlsbad, Calif.) in PBS. To eliminate remaining red blood cells, the digested sample can be washed (e.g. with 10% fetal bovine serum), treated with 160 mmol/L NH₄Cl, and finally suspended in DMEM complete medium (DMEM containing 10% FBS, 2 mmol/L glutamine and 1% penicillin/streptomycin). The cells can be filtered through a 40 μm nylon mesh.

Cultured human ASCs according to certain embodiments of the invention are described in DelaRosa et al. (Tissue Eng Part A. (2009) 15(10): 2795-806), Lopez-Santalla et al. (Stem cells (2015) 33: 3493-3503). In one embodiment (as described in Lopez-Santalla et al. 2015), human adipose tissue aspirates from healthy donors were washed twice with phosphate-buffered saline and digested with 0.075% collagenase (Type I; Invitrogen). The digested sample was washed with 10% fetal bovine serum (FBS), treated with 160 mM NH₄Cl to eliminate the remaining erythrocytes, and suspended in culture medium (Dulbecco's modified Eagle's medium (DMEM) with 10% FBS). Cells were seeded (2-3·10⁴ cells/cm²) in tissue culture flasks and cultured (37° C., 5% CO₂) with change of culture medium every 3-4 days. Cells were transferred to a new flask (10³ cells/cm²) when they reached 90% confluence. Cells were expanded up to duplication 12-14 and frozen. Experiments were performed with cells from two male and two female adult donors at population doublings 12-14. ASCs were thawed from the same cryobanks and seeded before each experiment. ASCs were defined according to the criteria of the International Society for Cellular Therapy: being positive for HLA-I, CD73, CD90, and CD105 and negative for CD11b, CD14, CD31, CD34, and CD45.

In another embodiment (as described by DelaRosa et al. 2009), lipoaspirates obtained from human adipose tissue from healthy adult donors were washed twice with PBS, and digested at 37° C. for 30 minutes with 18 U/mL of collagenase type I in PBS. One unit of collagenase liberates 1 mM of L-leucine equivalents from collagen in 5 hours at 37° C., pH 7.5 (Invitrogen, Carlsbad, Calif.). The digested sample was washed with 10% of fetal bovine serum (FBS), treated with 160 mM NH₄Cl, suspended in culture medium (DMEM containing 10% FBS), and filtered through a 40-mm nylon mesh. Cells were seeded (2-3×10⁴ cells/cm²) onto tissue culture flasks and Expanded at 37° C. and 5% CO₂, changing the culture medium every 7 days. Cells were passed to a new culture flask when cultures reached 90% of confluence. Cells were phenotypically characterized by their capacity to differentiate into chondro-, osteo-, and adipo-genic lineages. In addition, hASCs were verified by staining with specific surface markers. hASCs were positive for HLA-I, CD90, and CD105, and negative for HLA-II, CD40, CD80, CD86, and CD34. A pool from six healthy donors (three men and three women, aged between 35 and 47) was used in the study. Cells were used at passages 4-6.

The ASCs are cultured in a suitable tissue culture vessel, comprising a surface suitable for the adherence of ASCs e.g. plastic. Non-adherent cells are removed e.g. by washing in a suitable buffer, to provide an isolated population of adherent stromal cells (e.g. ASC). Cells isolated in this way can be seeded (preferably 2-3×10⁴ cells/cm²) onto tissue culture flasks and expanded at 37° C. and 5% CO₂, changing the culture medium every 3-4 days. Cells are preferably deattached from the adherent surface (e.g. by means of trypsin) and passed (“passaged”) to a new culture flask (1,000 cells/cm²) when cultures reach around 90% of confluence.

The ASCs may be cultured for at least about 15, at least about 20 days, at least about 25 days, or at least about 30 days. Typically the expansion of cells in culture improves the homogeneity of the cell phenotype in the population, such that a substantially pure population is obtained.

In some embodiments, the ASCs are expanded in culture for at least three culture passages or “passaged at least three times.” In other embodiments, the cells are passaged at least four times, at least five times, at least six times, at least seven times, at least eight times, at least nine times, or at least ten times. It is preferable that cells are passaged more than three times to improve the homogeneity of the cell phenotype in the cell population. Indeed, the cells may be expanded in culture indefinitely so long as the homogeneity of the cell phenotype is improved and differential capacity is maintained.

In some embodiments, the ASC are multiplied in culture for at least three population doublings, for example, the cells are expanded in culture for at least four, five, six, seven, eight, nine, ten, 15 or 20 population doublings. In some embodiments, the cells are expanded in culture for less than seven, eight, nine, ten, 15 or 20 population doublings. In certain embodiments, the cells are expanded in culture for between about 5 and 10 population doublings. In certain embodiments, the cells are expanded in culture for between about 10 and 15 population doublings. In certain embodiments, the cells are expanded in culture for between about 15 and 20 population doublings, for example about 16 population doublings.

ASC isolation is preferably carried out under sterile or GMP conditions.

The population of stem cells (e.g. ASCs) may be allogenic, i.e. not isolated from the subject into which the population of stem cells will be administered as a therapy.

### Populations of Stem Cells

Pretreatment with NAC, post-thaw treatment with NAC or a combination of both pretreatment and post-thaw treatment with NAC according to the methods disclosed herein may result in one or more, two or more, three or more, or all four of the following properties: increased viable cell number, increased growth rate, increased mitochondrial activity and improved recovery rate, as compared to a control population of stem cells. A control population of stem cells is the same population of stem cells that has not been pretreated with NAC, post-thaw treated with NAC or both, but has otherwise been subjected to identical conditions. In another embodiment, the control population of stem cells is derived from the same population of stem cells as the population of stem cells pretreated with NAC, post-thaw treated with NAC or both, but the control population has not been pretreated with NAC, post-thaw treated with NAC or both, but has otherwise been subjected to identical conditions.

Also provided is a population of stem cells (e.g. ASCs) obtained by any one of the methods described herein that possesses one or more, two or more, three or more, or all four of these properties.

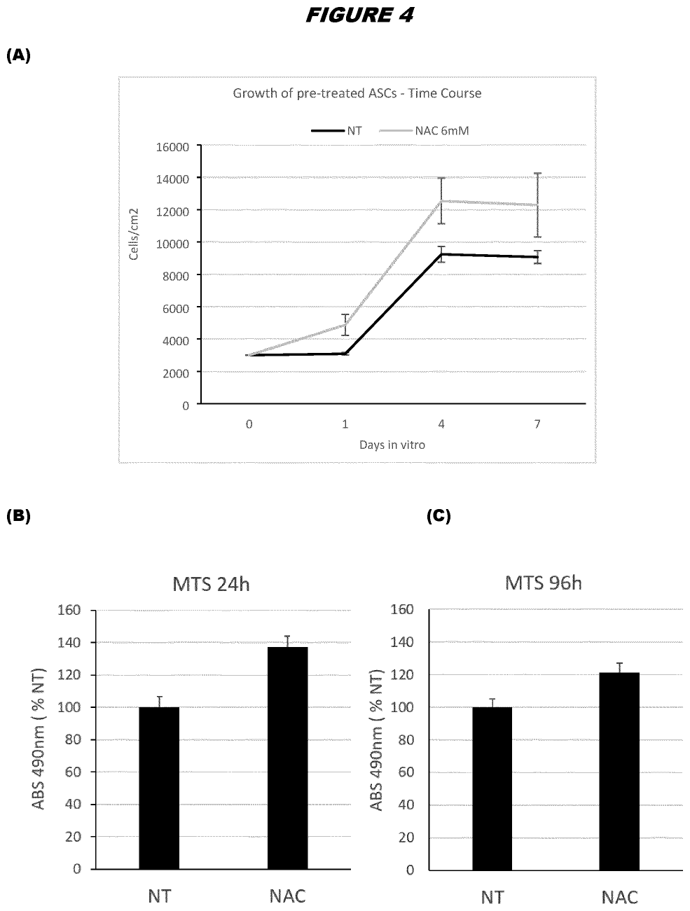

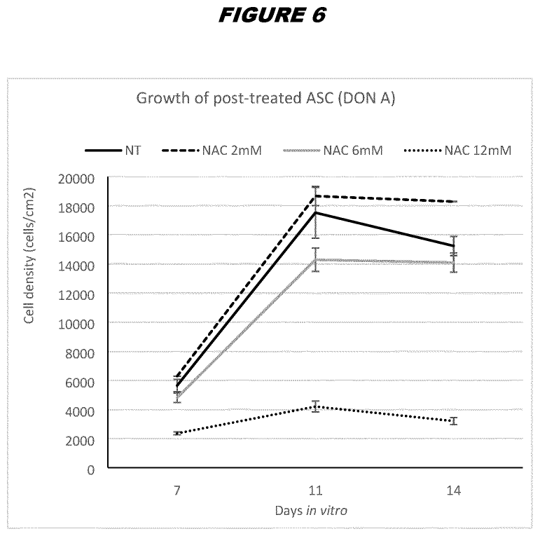

The number of viable cells following thaw, and optionally culture for about 1 day, about 2 days, about 3 days, about 4 days, about 7 days, or about 10 days or more, may be increased for the population of stem cells as compared to a control population of cells. For example, the number of viable cells after thaw and culture for 1 day (and/or 4 days) may be increased at least about 1.05-fold, at least about 1.1-fold, at least about 1.2-fold, at least about 1.3-fold, at least about 1.4-fold, at least about 1.5-fold, at least about 1.6-fold, at least about 2-fold, or at least about 5-fold or more in the population of stem cells as compared to a control population of stem cells. For example, FIG. 4A shows that the number of viable cells is increased for ASCs pretreated with 6 mM NAC relative to non-treated cells after 1 day of culture (˜5,000 vs ˜3,000 cells/cm²) and 4 days of culture (˜12,500 vs ˜9,000 cells/cm²). In another example, FIG. 6 shows that post-thaw treatment with 2 mM NAC increases the number of viable cells relative to non-treated cells at 7 (˜6,300 vs ˜5,600 cells/cm²), 11 (˜18,700 vs ˜17,500 cells/cm²) and 14 days (˜18,300 vs ˜15,200 cells/cm²) of culture. Suitable methods for measuring the number of viable cells are described above.

The growth rate of the population of stem cells (i.e. the increase in the number of viable cells/cm² per day) may be increased as compared to a control population of stem cells. The growth rate following thaw (e.g. between days 1 and 4 of post-thaw culture) may be increased at least about 1.03-fold, about 1.05-fold, at least about 1.1-fold, at least about 1.15-fold, at least about 1.2-fold, at least about 1.25-fold, at least about 1.3-fold, at least about 1.4-fold, at least about 1.5-fold, at least about 1.6-fold, or at least about 2-fold or more in the population of stem cells as compared to a control population of stem cells. For example, FIG. 4A shows that the rate of growth between days 1 and 4 of culture for ASCs pretreated with 6 mM NAC is increased relative to non-treated cells. Specifically, growth between days 1 and 4 for the NAC pretreated cells is approximately 2500 cells/cm²/day, compared to approximately 2000 cells/cm²/day for non-treated cells, i.e. an improvement of around 1.25-fold. In a further example, FIG. 6 shows that the rate of growth between days 7 and 11 of for ASCs post-thaw treated with 2 mM NAC is increased relative to non-treated cells, i.e. approximately 3100 cells/cm²/day, compared to approximately 3000 cells/cm²/day for non-treated cells.

The mitochondrial activity of the population of stem cells (as measured, e.g. by MTS assay) of the cells following thaw, and optionally culture for about 1 day, about 2 days, about 3 days, about 4 days, about 7 days, or about 10 days or more, may be increased as compared to a control population of stem cells. The mitochondrial activity after thaw and culture for 1 day (and/or 4 days) may be increased at least about 5%, at least about 10%, at least about 15%, at least about 20%, at least about 30%, at least about 35%, at least about 40% or at least about 50% or more in the population of stem cells as compared to a control population of stem cells. For example, FIG. 4B shows a greater than 35% increase in mitochondrial activity after pretreatment with 6 mM NAC as compared to non-treated cells at measured after 24 hours culture post-thaw (MTS assay readout at 490 nm was normalised at 100% for the control). In another example, FIG. 4C shows an increase of greater than 15% increase in mitochondrial activity after pretreatment with 6 mM NAC as compared to non-treated cells at measured after 96 hours culture post-thaw.

For adherent cells (such as ASCs), post-thaw “recovery” can be defined as the point when the viable cell number of adhered cells increases over the initial seeding density during culture. For cells that are grown in suspension, post-thaw “recovery” can be defined as when the viable cell number increases over the initial seeding density during culture. The recovery rate for the thawed population of stem cells, i.e. the time taken post-thaw for the cells to recover, may be improved (i.e. shortened) as compared to a control population of stem cells. For example, the number of hours taken to recover post-thaw may be decreased at least about 1.1-fold, at least about 1.2-fold, at least about 1.4-fold, at least about 1.6-fold, at least about 2-fold, at least 3-fold, at least 4-fold or at least 5-fold or more as compared to a control population of stem cells. For example, FIG. 4A shows that ASCs pretreated with 6 mM NAC are recovered after 1 day of post-thaw culture, while non-treated cells are not.

In preferred methods or populations of stem cells as disclosed herein, the population of stem cells possesses one or more, two or more, three or more, four or more, or all five of the following properties: (a) the number of viable cells following thaw and optionally culture for about 1 day and/or about 4 days is increased at least about 1.05-fold, at least about 1.1-fold, at least about 1.2-fold, at least about 1.3-fold, at least about 1.4-fold, at least about 1.5-fold, at least about 1.6-fold, at least about 2-fold or at least about 5-fold or more as compared to a control population of stem cells; (b) the growth rate (e.g. between days 1 and 4 of post-thaw culture) following thaw is increased at least about 1.03-fold, about 1.05-fold, at least about 1.1-fold, at least about 1.15-fold, at least about 1.2-fold, at least about 1.25-fold, at least about 1.3-fold, at least about 1.4-fold, at least about 1.5-fold, at least about 1.6-fold, or at least about 2-fold or more in the population of stem cells as compared to a control population of stem cells; (c) the mitochondrial activity following thaw and optionally culture for about 1 day and/or about 4 days is increased at least about 5%, at least about 10%, at least about 15%, at least about 20%, at least about 30%, at least about 35%, at least about 40% or at least about 50% as compared to a control population of stem cells; (d) the time taken post-thaw for the cells to recover is decreased as compared to a control population of stem cells; and/or (e) the number of hours taken for the cells to recover post-thaw is decreased at least about 1.1-fold, at least about 1.2-fold, at least about 1.4-fold, at least about 1.6-fold, at least about 2-fold, at least about 3-fold, at least about 4-fold, or at least about 5-fold relative to a control population of stem cells

In preferred methods or populations of stem cells as disclosed herein, the population of ASCs possess one or more, two or more, three or more, four or more, five or more, or all six of the following properties: (1) the number of viable cells following thaw and culture for about 1 day is increased at least about 1.5-fold as compared to a control population of stem cells (e.g. after pretreatment with 6 mM NAC for 24 hours); (2) the number of viable cells following thaw and culture for about 4 days at least about 1.3-fold as compared to a control population of stem cells (e.g. after pretreatment with 6 mM NAC for 24 hours); (3) the growth rate between days 1 and 4 of post-thaw culture is increased at least about 1.25-fold as compared to a control population of stem cells (e.g. after pretreatment with 6 mM NAC for 24 hours); (4) the mitochondrial activity following thaw and culture for about 1 day is increased at least about 35% as compared to a control population of stem cells (e.g. after pretreatment with 6 mM NAC for 24 hours); (5) the mitochondrial activity following thaw and culture for about 4 days is increased at least about 15% as compared to a control population of stem cells (e.g. after pretreatment with 6 mM NAC for 24 hours); and/or (6) the time taken post-thaw for the ASCs to recover is decreased as compared to a control population of stem cells (e.g. after pretreatment with 6 mM NAC for 24 hours).

In preferred methods or populations of stem cells as described herein, the population of ASCs possess one or more, two or more, three or more, or all four of the following properties: (a) the number of viable ASCs following post-thaw treatment with NAC (e.g. 2 mM) for 7 days is increased at least about 1.1-fold as compared to a control population of stem cells; (b) the number of viable ASCs following post-thaw treatment with NAC (e.g. 2 mM) for 11 days is increased at least about 1.05-fold as compared to a control population of stem cells; (c) the number of viable ASCs following post-thaw treatment with NAC (e.g. 2 mM) for 14 days is increased at least about 1.2-fold as compared to a control population of stem cells; and/or (d) the growth rate following post-thaw treatment with NAC (e.g. 2 mM) is increased at least about 1.03-fold as compared to a control population of stem cells when measured between days 7 and 11 of culture.

### Cryopreservation Compositions

Disclosed is a cryopreservation composition comprising the population of stem cells (e.g. ASCs) made by any one of the methods disclosed herein and a cryopreservation medium. The cryopreservation composition may be frozen. The cryopreservation composition may contain NAC, for example at a concentration in the range of around 0.5-10 mM, for example, around 2-8 mM or around 4-6 mM. In a particularly preferred embodiment, the concentration of NAC in the cryopreservation composition is about 6 mM.

In practicing the methods of the invention, it is envisioned that the cryopreservation process may have an effect on a variety of cellular processes. As discussed above, the freezing process may halt intracellular reactions, including gene transcription. These effects may also result from, or in addition to, chemical composition of the cryopreservation medium (such as metabolic effects of the cryoprotectant, ion concentrations) or the pretreatment of the cells with NAC. Also, in cryopreservation, the stresses induced by freezing affect cellular transport processes involving heat shock or membrane destabilization proteins.

### Pharmaceutical Compositions

Disclosed is a pharmaceutical composition comprising the population of stem cells (e.g. ASCs) of made by any one of the methods disclosed herein and a pharmaceutically acceptable carrier.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Examples of a pharmaceutically acceptable carrier include a pharmaceutically acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

The pharmaceutical composition may be sterile, free of the presence of unwanted virus, bacteria and other pathogens, as well as pyrogen-free. That is, for human administration, the subject compositions should meet sterility, pyrogenicity as well as general safety and purity standards as required by FDA Office of Biologics standards.

Because of difficulties in obtaining sufficient autologous stem cells, the population of stem cells disclosed herein may be obtained from an allogeneic source. It is known in the art that bone marrow-derived MSCs and ASCs do not provoke a response of allogeneic lymphocytes in vitro and consequently, these cells can be used for any patient, irrespective of MHC incompatibility. Thus, the population of stem cells (e.g. the bone marrow-derived MSCs or ASCs) in the pharmaceutical composition may be allogeneic with respect to the intended transplantation host.

The pharmaceutical composition may comprise a suspension of the population of stem cells in various solutions or materials, e.g. for use as pharmaceuticals or biomaterials, as described in more detail below. The pharmaceutical composition may comprise a suspension of stem cells (e.g. allogeneic ASCs) in Ringer's solution and HSA. The pharmaceutical composition may comprise a suspension of the stem cells (e.g. allogeneic ASCs) in aseptic buffered saline solution. The cells may be provided in disposable vials without preservative agents. The cells can be given at a dose of 120 million cells (e.g. at a concentration of 5 million cells/mL). The cells (e.g. ASCs) can also be administered at around 1 million to 10 million cells/kg

In certain embodiments, the pharmaceutical composition is a suspension of the stem cells (e.g. allogeneic ASCs) in a material, such as a polymer, glue, gel, etc. Such suspensions may be prepared, for example, by sedimenting out the stem cells from the culture medium and re-suspending them in the desired solution or material. The cells may be sedimented and/or changed out of the culture medium, for example, by centrifugation, filtration, ultrafiltration, etc.

The concentration of the subject adipose tissue-derived stromal stem cells in the subject adipose tissue-derived stromal stem cell-containing compositions may be at least about 5 million cells/mL, at least about 10 million cells/mL, at least about 20 million cells/mL, at least about 30 million cells/mL, or at least about 40 million cells/mL. Typically the concentration between about 1 million cells/mL and 10 million cells/mL, e.g. between about between about 5 million cells/mL and 10 million cells/mL. In certain embodiments, the cell density is around 5 million cells/mL in pharmaceutical composition.

In certain embodiments, the pharmaceutical composition comprises around 1 million to 150 million cells, preferably around 30 million cells or around 120 million cells.

In some instances, the pharmaceutical composition may comprise NAC. In other instances, the pharmaceutical composition may not comprise NAC.

Pharmaceutically acceptable carriers and diluents include saline, aqueous buffer solutions, solvents and/or dispersion media. The use of such carriers and diluents is well known in the art. The solution is typically sterile and fluid to the extent that easy syringability exists. Typically, the solution is stable under the conditions of manufacture and storage and preserved against the contaminating action of microorganisms such as bacteria and fungi through the use of, for example, parabens, chlorobutanol, phenol, ascorbic acid, thimerosal, and the like. The pharmaceutical composition may be prepared by suspending the population of stem cells (e.g. ASCs) as described herein in a pharmaceutically acceptable carrier or diluent and, as required, other ingredients enumerated above, followed by filtered sterilization.

Some examples of materials and solutions which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances typically employed in pharmaceutical formulations.

In certain embodiments, the pharmaceutical composition further comprises an adhesive. The adhesive may be a fibrin-based adhesive, such as a fibrin gel or fibrin glue or fibrin-based polymer or adhesive, or other tissue adhesive or surgical glue, such as, for example cyanoacrylate, collagen, thrombin, and polyethylene glycol. Other materials that may be used include but are not limited to calcium alginate, agarose, types I, II, IV or other collagen isoform, poly-lactic/poly-glycolic acid, hyaluronate derivatives or other materials (Perka et al. J. Biomed. Mater. Res. (2000) 49: 305-311; Sechriest et al. J. Biomed. Mater. Res. (2000) 49: 534-541; Chu et al. J. Biomed. Mater. Res. (1995) 29:1147-1154; Hendrickson et al. Orthop. Res. (1994) 12: 485-497). In other embodiments, the adhesive is a liquid bandage, wherein population of stem cells (e.g. ASCs) is mixed with the liquid bandage material. A “liquid bandage” is a solution comprising a compound, e.g. a polymeric material, which is applied to a wound with a spray or a brush, followed by removing the solvent by vaporization to provide a protective film on the wound.

The pharmaceutical composition may also be used to coat a support, e.g. a medical device. For example, the support may be a suture or thread. The support may be coated with cells in any way as known to one of skill in the art, e.g. by soaking, spraying, painting, imprinting, etc. In one embodiment, the support is a suture, staple, absorbable thread, non-absorbable thread, natural thread, synthetic thread, monofilament thread or multifilament thread (also called braids). Preferred methods of preparing sutures and other supports used to close wounds coated with adipose tissue-derived stromal stem cells are disclosed in U.S. patent application Ser. No. 11/056,241 “Biomaterial for Suturing”, filed Feb. 14, 2005, which application is incorporated by reference in its entirety. The pharmaceutical composition disclosed herein represent novel compositions that may be used with the methods disclosed in U.S. patent application Ser. No. 11/056,241.

Further, in any of the disclosed pharmaceutical compositions, at least one therapeutic agent may be incorporated into the composition (although not required and can optionally be excluded). For example, the pharmaceutical composition may contain an analgesic (e.g. to aid in treating inflammation or pain), or an anti-infective agent to prevent infection of the site treated with the composition.

More specifically, non-limiting examples of useful therapeutic agents that may be included in the pharmaceutical composition described herein include the following therapeutic categories: analgesics, such as nonsteroidal anti-inflammatory drugs, opiate agonists and salicylates; anti-infective agents, such as antihelmintics, antianaerobics, antibiotics, aminoglycoside antibiotics, antifungal antibiotics, cephalosporin antibiotics, macrolide antibiotics, miscellaneous ß-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, anti-inflammatory agents, corticosteroid anti-inflammatory agents, antipruritics/local anesthetics, topical anti-infectives, antifungal topical anti-infectives, antiviral topical anti-infectives; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hormones and hormone modifiers, such as abortifacients, adrenal agents, corticosteroid adrenal agents, androgens, anti-androgens, immunobiologic agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immunosuppressive anti-inflammatory agents, nonsteroidal anti-inflammatory drugs (NSAIDs), salicylate anti-inflammatory agents, minerals; and vitamins, such as vitamin A, vitamin B, vitamin C, vitamin D, vitamin E, and vitamin K.

Preferred classes of useful therapeutic agents from the above categories include: (1) analgesics in general, such as lidocaine or derivatives thereof, and nonsteroidal anti-inflammatory drugs (NSAIDs) analgesics, including diclofenac, ibuprofen, ketoprofen, and naproxen; (2) opiate agonist analgesics, such as codeine, fentanyl, hydromorphone, and morphine; (3) salicylate analgesics, such as aspirin (ASA) (enteric coated ASA); (4) H₁-blocker antihistamines, such as clemastine and terfenadine; (5) anti-infective agents, such as mupirocin; (6) antianaerobic anti-infectives, such as chloramphenicol and clindamycin; (7) antifungal antibiotic anti-infectives, such as amphotericin b, clotrimazole, fluconazole, and ketoconazole; (8) macrolide antibiotic anti-infectives, such as azithromycin and erythromycin; (9) miscellaneous ß-lactam antibiotic anti-infectives, such as aztreonam and imipenem; (10) penicillin antibiotic anti-infectives, such as nafcillin, oxacillin, penicillin G, and penicillin V; (11) quinolone antibiotic anti-infectives, such as ciprofloxacin and norfloxacin; (12) tetracycline antibiotic anti-infectives, such as doxycycline, minocycline, and tetracycline; (13) antituberculosis antimycobacterial anti-infectives such as isoniazid (INH), and rifampin; (14) antiprotozoal anti-infectives, such as atovaquone and dapsone; (15) antimalarial antiprotozoal anti-infectives, such as chloroquine and pyrimethamine; (16) anti-retroviral anti-infectives, such as ritonavir and zidovudine; (17) antiviral anti-infective agents, such as acyclovir, ganciclovir, interferon alfa, and rimantadine; (18) antifungal topical anti-infectives, such as amphotericin B, clotrimazole, miconazole, and nystatin; (19) antiviral topical anti-infectives, such as acyclovir; (20) electrolytic and renal agents, such as lactulose; (21) loop diuretics, such as furosemide; (22) potassium-sparing diuretics, such as triamterene; (23) thiazide diuretics, such as hydrochlorothiazide (HCTZ); (24) uricosuric agents, such as probenecid; (25) enzymes such as RNase and DNase; (26) antiemetics, such as prochlorperazine; (27) salicylate gastrointestinal anti-inflammatory agents, such as sulfasalazine; (28) gastric acid-pump inhibitor anti-ulcer agents, such as omeprazole; (29) H₂-blocker anti-ulcer agents, such as cimetidine, famotidine, nizatidine, and ranitidine; (30) digestants, such as pancrelipase; (31) prokinetic agents, such as erythromycin; (32) ester local anesthetics, such as benzocaine and procaine; (33) musculoskeletal corticosteroid anti-inflammatory agents, such as beclomethasone, betamethasone, cortisone, dexamethasone, hydrocortisone, and prednisone; (34) musculoskeletal anti-inflammatory immunosuppressives, such as azathioprine, cyclophosphamide, and methotrexate; (35) musculoskeletal nonsteroidal anti-inflammatory drugs (NSAIDs), such as diclofenac, ibuprofen, ketoprofen, ketorlac, and naproxen; (36) minerals, such as iron, calcium, and magnesium; (37) vitamin B compounds, such as cyanocobalamin (vitamin B₁₂) and niacin (vitamin B₃); (38) vitamin C compounds, such as ascorbic acid; and (39) vitamin D compounds, such as calcitriol.

In certain embodiments, the therapeutic agent may be a growth factor or other molecule that affects cell differentiation and/or proliferation. Growth factors that induce final differentiation states are well-known in the art, and may be selected from any such factor that has been shown to induce a final differentiation state. Growth factors for use in methods described herein may, in certain embodiments, be functional variants or fragments of a naturally-occurring growth factor. For example, a variant may be generated by making conservative amino acid changes and testing the resulting variant testing for growth factor function using an assay known in the art.

### Uses and Applications

### Use of NAC

Disclosed is the use of NAC for the cryopreservation of stem cells, for example, in any one of the methods disclosed herein.

### Medical Applications

Stem cells are being used to treat an expanding number of disease and disorders. Thus, the population of stem cells made according to any one of the methods disclosed herein, the pharmaceutical composition as disclosed herein or a cryopreservation composition as disclosed herein may be used in therapy. The term “therapy” is intended to cover treatment and/or prevention of a disease, disorder or symptom in a patient. The terms “subject”, “recipient” and “patient” are used interchangeably herein and refer unless explicitly stated to any human or non-human animal (e.g. a mammal) in need of therapy. In preferred embodiments, the patient is a human. When the patient is a human, the population of stem cells is generally human.

Disclosed is a population of stem cells, a pharmaceutical composition or a cryopreservation composition as described herein for use in a method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection in a patient in need thereof. The population of cells used in the method may be made by any one of the methods for stem cell cryopreservation disclosed herein.

Also disclosed is the use of a population of stem cells, a pharmaceutical composition or a cryopreservation composition as described herein for the manufacture of a medicament for treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection in a patient in need thereof.

Further disclosed is a method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection, the method comprising administering a population of stem cells, a pharmaceutical composition or a cryopreservation composition as disclosed herein to a subject in need thereof.

The population stem cells, pharmaceutical composition or cryopreservation composition as described herein, in particular when the population of stem cells are ASCs, may be used to treat fistula. The term “fistula” refers to any abnormal passage or communication or connection, usually between two internal organs or leading from an internal organ to the surface of the body, e.g. a connection or passageway between organs or vessels that normally do not connect. For example, types of fistulae, named for the areas of the body in which they occur, include anorectal fistula or fistula-in-ano or fecal fistula (between the rectum or other anorectal area and the skin surface), arteriovenous fistula or A-V fistula (between an artery and vein), biliary fistula (between the bile ducts to the skin surface, often caused by gallbladder surgery), cervical fistula (abnormal opening in the cervix), craniosinus fistula (between the intracranial space and a paranasal sinus), enteroenteral fistula (between two parts of the intestine), enterocutaneous fistula (between the intestine and the skin surface, namely from the duodenum or the jejunum or the ileum), enterovaginal fistula (between the intestine and the vagina), gastric fistula (between the stomach to the skin surface), metroperitoneal fistula (between the uterus and peritoneal cavity), perilymph fistula (a tear between the membranes between the middle and inner ears), pulmonary arteriovenous fistula (between an artery and vein of the lungs, resulting in shunting of blood), rectovaginal fistula (between the rectum and the vagina), umbilical fistula (between the umbilicus and gut), tracheoesophageal fistula (between the breathing and the feeding tubes) and vesicovaginal fistula (between the bladder and the vagina). Causes of fistulae include trauma, complications from medical treatment and disease. Inflammatory bowel diseases, such as Crohn's disease and ulcerative colitis, are the leading causes of anorectal, enteroenteral, and enterocutaneous fistulae. In certain embodiments, the fistula is a perianal fistula, for example, refractory complex perianal fistulas in patients with Crohn's Disease. The population of stem cells (e.g. allogeneic ASCs) may be administered at a dose of about 120 million cells (e.g. about 5 million cells/mL) for intralesional injection.

Disclosed is a population of stem cells as disclosed herein for use in a method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection, in a patient in need thereof, wherein the method comprises the steps of: (a)

treating a population of stem cells with NAC to obtain a treated population of stem cells; (b) freezing the treated population of stem cells to obtain a frozen population of stem cells; (c) thawing the frozen population of stem cells to obtain a thawed population of stem cells; (d) optionally culturing the thawed population of stem cells to obtain an expanded population of stem cells; and (e) administering the population of stem cells to the patient.

Also disclosed is the use of a population of stem cells as disclosed herein for the manufacture of a medicament for treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection, in a patient in need thereof, wherein the method comprises the steps of: (a) treating a population of stem cells with NAC to obtain a treated population of stem cells; (b) freezing the treated population of stem cells to obtain a frozen population of stem cells; (c) thawing the frozen population of stem cells to obtain a thawed population of stem cells; (d) optionally culturing the thawed population of stem cells to obtain an expanded population of stem cells; and (e) administering the population of stem cells to the patient.

Further disclosed is a method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection, in a patient in need thereof, the method comprising the steps of: (a) treating a population of stem cells with NAC to obtain a treated population of stem cells; (b) freezing the treated population of stem cells to obtain a frozen population of stem cells; (c) thawing the frozen population of stem cells to obtain a thawed population of stem cells; (d) optionally culturing the thawed population of stem cells to obtain an expanded population of stem cells; and (e) administering the population of stem cells to the patient.

In certain embodiments, the method of treatment and/or prevention further comprises any one of the steps as defined in the methods disclosed herein (e.g. “pretreatment”) prior to administration of the population of stem cells to the patient.

Disclosed is a population of stem cells as described herein for use in a method of treating fistula and/or treating and/or preventing an inflammatory disorder, an autoimmune disease or an immunologically-mediated disease, such as sepsis, rheumatoid arthritis, allergies (e.g. hypersensitivity Type IV reactions), irritable bowel disease, Crohn's disease, ulcerative colitis or organ rejection in a patient in need thereof, wherein the method comprises the steps of: (a) freezing a population of stem cells to obtain a frozen population of stem cells; (b) thawing the frozen population of stem cells to obtain a thawed population of stem cells; (c) culturing the thawed population of stem cells in the presence NAC to obtain an expanded population of stem cells; and (d) administering the population of stem cells to the patient.

Also disclosed is the use of a population of stem cells as described herein for the manufacture of a medicament for treating fistula and/or treating and/or preventing an inflammatory disorder, an autoimmune disease or an immunologically-mediated diseases, such as sepsis, rheumatoid arthritis, allergies (e.g. hypersensitivity Type IV reactions), irritable bowel disease, Crohn's disease, ulcerative colitis or organ rejection in a patient in need thereof, wherein the method comprises the steps of: (a) freezing a population of stem cells to obtain a frozen population of stem cells; (b) thawing the frozen population of stem cells to obtain a thawed population of stem cells; (c) culturing the thawed population of stem cells in the presence NAC to obtain an expanded population of stem cells; and (d) administering the population of stem cells to the patient.

Further disclosed is a method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection, in a patient in need thereof, the method comprising the steps of: (a) freezing a population of stem cells to obtain a frozen population of stem cells; (b) thawing the frozen population of stem cells to obtain a thawed population of stem cells; (c) culturing the thawed population of stem cells in the presence NAC to obtain an expanded population of stem cells; and (d) administering the population of stem cells to the patient.

In certain embodiments, the method of treatment and/or prevention further comprises any one of the steps as defined in the methods disclosed herein (e.g. “post-thaw treatment”) prior to administration of the population of stem cells to the patient.

The population of stem cells, pharmaceutical composition or cryopreservation composition may be administered at a dose of between around 1 and 150 million stem cells (e.g. allogeneic ASCs). In preferred embodiments, the stem cells (e.g. allogeneic ASCs) may be administered in a dose of around 30 million or around 120 million cells.

Administration of the population of stem cells, pharmaceutical compositions or cryopreservation compositions as disclosed herein to subjects, particularly human subjects, may be carried out by injection or implantation of the cells into target sites in the subjects. For example, a delivery device which facilitates introduction by, injection or implantation, into the subject may be used. Such delivery devices include tubes, e.g., catheters, for injecting into the body of a recipient subject. In a preferred embodiment, the tubes additionally have a needle, e.g., a syringe, through which the population of stem cells, pharmaceutical compositions or cryopreservation compositions can be introduced into the subject at a desired location.

In preferred embodiments, the population of stem cells—including those in the pharmaceutical compositions and/or cryopreservation compositions—are ASCs.

The stem cells may be allogeneic or autologous.

Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. Although compounds that exhibit toxic side effects may be used, care should be taken to design a delivery system that targets the agents to the desired site in order to reduce side effects.

The data obtained from the cell culture assays and animal studies may be used in formulating a range of dosage for use in humans. The dosage of any therapeutic agent or alternatively of any components therein, lies typically within a range of circulating concentrations that include the ED₅₀ with little or no toxicity. The dosage may vary within this range depending upon the dosage form employed and the route of administration utilized. For agents of the present invention, the therapeutically effective dose may be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information may be used to more accurately determine useful doses in humans. Levels in plasma may be measured, for example, by high performance liquid chromatograph

### Kits

Disclosed is a cryopreservation kit comprising: a cryovial, a container containing NAC and a container comprising a population of stem cells. The kit may comprise instructions for its use. Disclosed is a cryopreservation kit comprising: a plurality of cryovials, a container containing NAC and a container comprising a population of stem cells. The population of stem cells may be provided in the kit as a compositions or pharmaceutical compositions as disclosed herein.

### General Definitions

Unless defined otherwise, all technical and scientific terms used herein have the same meaning as commonly understood to one of ordinary skill in the art to which this invention belongs.

The articles “a” and “an” refer to one or to more than one (i.e., to at least one) of the grammatical object of the article. By way of example, “an element” means one element or more than one element.

The terms “comprise” and “comprising” are used in the inclusive, open sense, meaning that additional elements may be included.

In general, methods “comprising” a number of steps do not require the steps to be performed in a particular order. Where a method comprises a number of sequentially numbered or alphabetical steps (e.g. (1), (2), (3) or (a), (b), (c) etc.), this implies that the steps must be performed in the prescribed order unless stated otherwise. Such language does not, however, exclude the possibility of additional steps being performed in between each of the prescribed steps.

The term “including” is used herein to mean “including but not limited to”. “Including” and “including but not limited to” are used interchangeably.

### EXAMPLES

The invention now being generally described, it will be more readily understood by reference to the following examples, which are included merely for purposes of illustration of certain aspects and embodiments of the present invention, and are not intended to limit the invention.

### Example 1—ASC Isolation and Culture

Human samples were obtained with informed consent (as approved by the Spanish Ethics Committee of reference for the site of tissue procurement; Clinica de la Luz Hospital, Madrid, Spain). ASCs were obtained as previously published (Mancheno-Corvo et al., Frontiers in Immunology (2017), 8, 462; Menta et al., Frontiers in Immunology (2014), 8, 462). Briefly, human adipose tissue aspirates from healthy donors were washed twice with phosphate-buffered saline (PBS) and digested with 0.075% collagenase (Type I, Invitrogen, Carlsbad, Calif., USA). The digested sample was washed with 10% fetal bovine serum (FBS), treated with 160 mM NH₄Cl to eliminate remaining erythrocytes and suspended in culture medium (Dulbecco's Modified Eagle Medium (DMEM), with 10% FBS). Cells were seeded in tissue culture flasks and expanded (37° C., 5% CO₂) with change of culture medium every 3-4 days. Cells were transferred to a new flask when they reached 90% confluence. Cells were expanded up to duplication 12-14 and frozen in FBS with 10% DMSO (FBS with 10% DMSO was used as the freezing medium when freezing the ASCs throughout all the examples described herein). Experiments were performed with a pool of cells from three male and three female adult donors at population doublings 12-14. The expanded ACSs (eASCs) were confirmed to meet the definition according to the criteria of the International Society for Cellular Therapy (Dominici et al., Cytotherapy (2006) 8(4): 315-317), being positive for CD73 (AD2) and CD90 (5E10) from Becton Dickinson (Franklin Lakes, N.J., USA) and CD105 (43A3) from Biolegend (San Diego, Calif., USA) and negative for CD14 (RM052) from Immunotech (Monrovia, Calif., USA), CD19 (4G7), HLA-DR (L243), and CD34 (8G12) from Becton Dickinson and CD45 (J33) from Beckman Coulter (Brea, Calif., USA).

### Example 2—Assessing Various Pretreatment Steps on Post-Thaw ASC Cell Number

### ASC Pretreatment

ASCs from donor A were thawed by warming the vials in a 37° C. bath and diluting the freezing medium containing DMSO with fresh complete DMEM (DMEM/F-12 media—GlutaMAX™-I, Gibco, supplemented with 100 μg/mL penicillin/streptomycin and 10% FBS). Cells were centrifuged at 450 g for 6 minutes at room temperature to eliminate leftover DMSO and plated in T-175 flasks at 20.000 cells/cm² in complete DMEM. 24 hours post-thaw, the cells were treated with the suitable concentrations of the compounds indicated in the table below for 24 hours:

| | Concentration | | |
|---|---|---|---|
| Compound | used herein | | Reference |
| NAC | 6 | mM | Li et al., Scientific Reports (2015) 5: 9819 |
| LY294 | 10 | μM | Gharibi et al., Stem Cells (2014) 32: 2256- |
| | | | 2266 |
| sc79 | 10 | μM | Chen et al. Oncotarget (2017) 8(19): |
| | | | 31065-31078 |
| Exendin-4 | 20 | nM | Zhou et al. Scientific Reports (2015) 5: |
| | | | 12898 & Zhou et al. Free Radical Biology |
| | | | and Medicine (2014) 77: 363-375 |

A 600 mM stock of NAC (SIGMA) was prepared in Milli-Q water (Millipore). This stock was used for pre-treatment and post-treatment by adding directly 50 μL stock solution per well into 5 mL of medium, making a final concentration of 6 mM. For 2 mM, only 16.7 μL were added per well, and in the case of 12 mM, 100 μL of stock were added. DMSO was used as the vehicle for sc79 and LY294.

Following the pretreatment step, the medium was removed, the cells were washed with PBS and trypsinized using trypsin-EDTA 0.25% (ThermoFisher) for 8 minutes at 37° C. After trypsin inactivation with complete DMEM, cells were harvested and centrifuged prior to resuspension in freezing medium (FBS with 10% DMSO), and frozen into 500,000 or 1 million cells per vial and stored in liquid nitrogen for further use. Specifically, the cells were frozen at −80° C. for 24 hours in a Cool Cell® device (BioCision) and then transferred to a liquid nitrogen storage container. All experiments were performed in incubators and 37° C., 5% CO₂.

### Assessing Various Pretreatment Steps on Post-Thaw Cell Number and Growth

ASC were seeded into 96-well flat bottom plates (1000 or 2000 ASC per well), cultured for 24 hours and then viable cell number was assessed using the MTS assay (CellTiter 96° Aqueous One Solution Cell Proliferation Assay; Promega) following the manufacturer's instructions. The CellTiter 96° Aqueous One Solution Cell Proliferation Assay is a colorimetric method for determining the number of viable cells. The CellTiter 96° Aqueous One Solution reagent contains a tetrazolium compound [3-(4,5-dimethylthiazol-2-yl)-5-(3-carboxymethoxyphenyl)-2-(4-sulfophenyl)-2H-tetrazolium, MTS] and an electron coupling reagent (phenazine ethosulfate; PES). The MTS tetrazolium compound is bioreduced by cells—presumably by NADPH or NADH produced by dehydrogenase enzymes in metabolically active cells—into a coloured formazan product that is soluble in tissue culture medium.

Briefly, 40 μL of the reagent was added to 200 μL of complete DMEM in each well and absorbance was measured after 2-3 hours at 490 nm using a Navision system (Microsoft). Each condition was measured in 6 technical repeats. The MTS assay results are presented as percentage of absorbance at 490 nm relative to the non-treated (NT) cells.

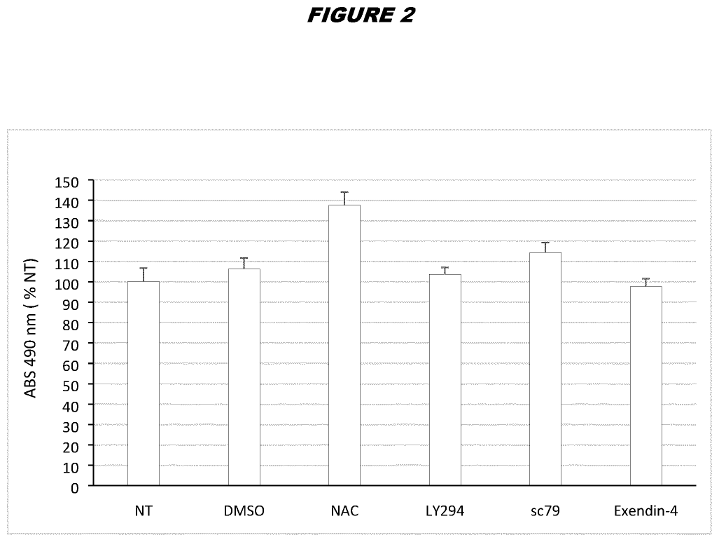

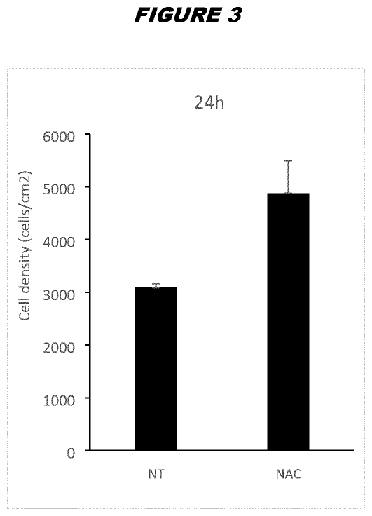

NAC pre-treatment resulted in increased cell numbers 24 hours post-seeding in comparison to non-treated (NT), as assessed by MTS assay (FIG. 2) and by cell density (FIG. 3).

In addition to NAC, Exendin-4, IL6, sc79 and LY294 pre-treatments were assessed. Sc79 is an activator of PI3K pathway, a proliferation and pro-survival signal (Jo et al. Proceedings of the National Academy of Sciences (2012), 109(26): 10581-10586, Chen et al. Oncotarget (2017) 8(19): 31065-31078), and LY294 is an inhibitor molecule of this same pathway used in Zonca et al., (2012) Tissue Engineering: Part A 18(7-8): 852-859 and Gharibi et al., (2014) Stem Cells 32: 2256-2266. DMSO was used as the vehicle for sc79 and LY294. The pre-treatment with compounds other than NAC did not show reproducible effects on cell number.

### Example 3—Further Assessing NAC Pre-Treatment Steps on Post-Thaw ASC Cell Number

### ASC Proliferation Assay

ASCs pretreated with NAC according to the methods described in Example 2 from donor A or donor B final drug substance (FDS) were thawed by warming the vials in a 37° C. bath and quickly diluting the freezing medium containing DMSO (FBS with 10% DMSO) with fresh complete DMEM. Cells were centrifuged at 450 g for 6 minutes at room temperature to eliminate leftover DMSO, and plated in P6-well plates (Falcon #353046) in triplicates at 3000 cells per well in 5 mL of complete DMEM per well. Cells were washed with 1×PBS and trypsinized using trypsin-EDTA 0.25% (ThermoFisher) for 8 minutes at 37° C. After trypsin inactivation using complete DMEM, cells were harvested, centrifuged and resuspended in fresh DMEM; triplicate wells were unified as a single sample for counting purposes. Cells were counted in triplicates at 24 hours, 96 hours and 7 days after plating using an Invitrogen Countess Automated Cell Counter (Invitrogen) and adding trypan blue as a viability stain (FIG. 4A). The calculation of cell density was done using the count of viable ASC (negative for trypan blue) per surface unit (cm²).

Pre-treating with NAC increased the number of cells counted at 24 hours and 4 days (12,500 cells as compared to 9,200 cells/cm² in the non-treated control at 4 days post-thawing, as shown in FIG. 4A). These findings were supported by MTS data performed in parallel, showing a 15-20% increase of mitochondrial activity after NAC pretreatment (FIGS. 4B & C). ASC growth had reached confluency before day 7, therefore there is no significant growth at this time point, as compared to day 4.

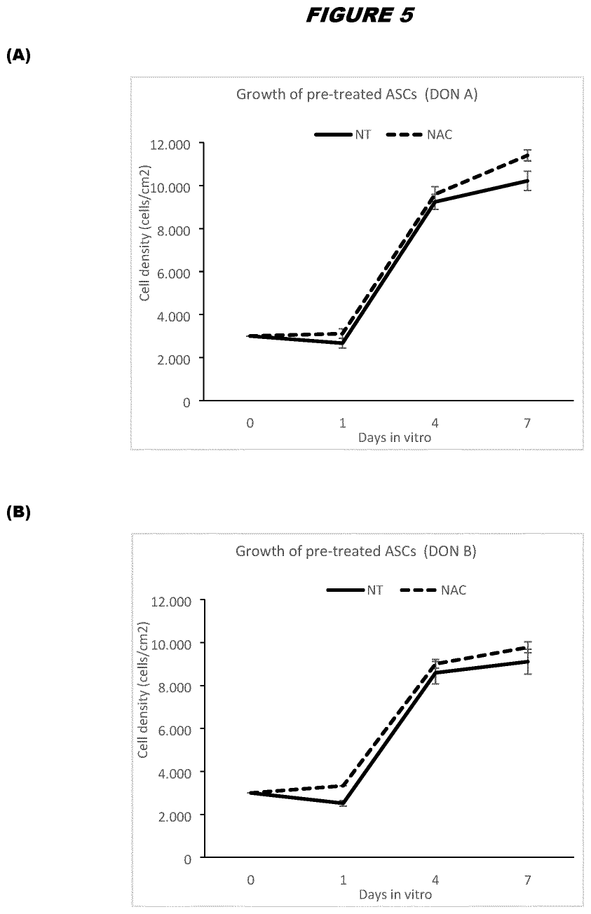

To reconfirm the data discussed above, growth assays were performed after NAC pretreatment with two different ASC donors (donor A (DON A) and donor B (DON B)). Cell numbers were analysed at day 1, 4 and 7 after thawing NAC pre-treated or non-treated cells for each donor (FIGS. 5A & B). Cells from both donors showed an increase in cell numbers from 24 hours post-seeding and this increase was maintained for a week in culture (FIG. 5). This data confirms that NAC pre-treatment increases the cell numbers after freeze-thaw recovery.

### Example 4—Effect of Post-Thaw Treatment with Different Concentrations of NAC on Cell Growth after Thawing

The effect of post-thaw treatment with different concentrations of NAC (post-thaw NAC treatment) on cell growth was also studied. ASCs were frozen (without NAC pretreatment), thawed as discussed above, and then treated with three different NAC concentrations (2, 6 and 12 mM) in complete DMEM prior to plating, and the cell numbers at day 4, 11 and 14 were analysed. Post-thaw treatment with 2 mM NAC resulted in an increase in cell number that was sustained up to 2 weeks in culture (FIG. 6). One possible explanation for the lower cell densities observed after post-thaw treatment with 6 mM and 12 mM NAC is that these concentrations of NAC may impact on the adherence of post-thaw (‘floating’) ASCs to the plate.

### Example 5—NAC Pretreatment does not Affect the Identity of ASCs after Thaw and Culture

The expression of four surface markers (CD29, CD73, CD90 and CD105, consistent with the criteria of the International Society for Cellular Therapy (Dominici et al., Cytotherapy (2006) 8(4): 315-317) were used to confirm the identity of thawed and expanded ASCs following pretreatment with NAC at concentration of 6 mM according to the methods described in Example 2.

The identity of the cells was analysed following standard protocols, after two weeks in culture (post-thawing). The harvested cells were stained with suitable concentrations of the antibodies indicated in the table below (diluted as per the manufacturers' instructions) and assessed using a FACSCalibur cytometer (BD).

| Marker | Antibody | Antibody source |
|---|---|---|
| CD29 | MAR4 | Becton Dickinson (Franklin |
| CD73 | AD2 | Lakes, NJ, USA) |
| CD90 | 5E10 | |
| CD105 | 43A3 | Biolegend (San Diego, CA, |
| | | USA) |

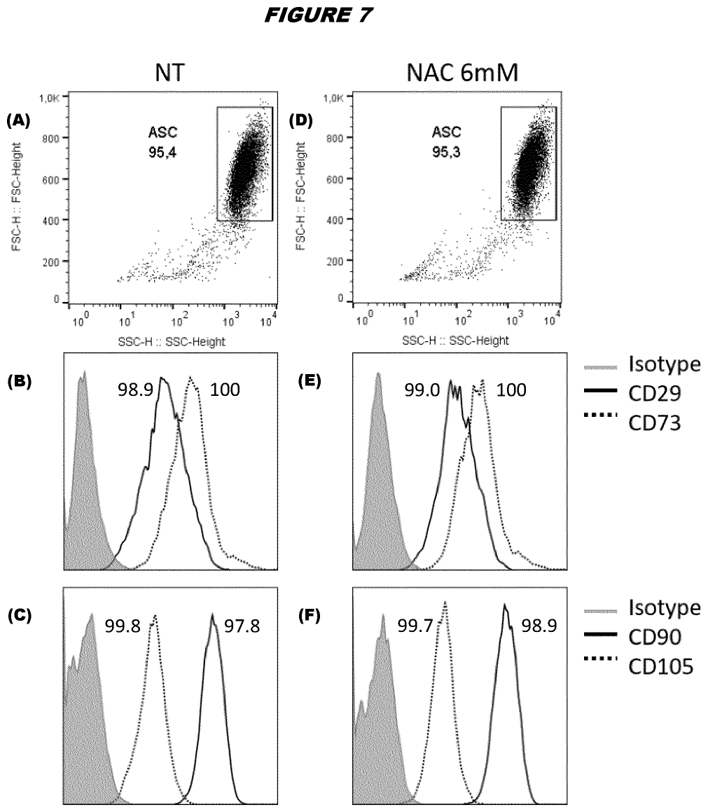

The data were analysed using FCS Express software. FIG. 7 shows that the cells express CD29, CD73, CD90 and CD105, and confirms that NAC pretreatment of cells prior to freezing does not alter the expression of ASC identity markers after thaw and culture.

### Example 6—NAC Pretreatment does not Significantly Affect the Capacity of Thawed ASCs to Inhibit the Proliferation of Stimulated Lymphocytes

After showing that pre-treatment of ASCs with NAC resulted in a growth advantage in vitro, experiments were conducted to see if NAC pretreatment affected ACS functional properties. First, the capacity of thawed and expanded ASCs (pretreated with NAC according to the methods in Example 2) to inhibit the proliferation of stimulated lymphocytes was measured.

As previously published for immunosuppression assays (Mancheño-Corvo et al., Frontiers in Immunology (2017), 8, 462; Menta et al., Frontiers in Immunology (2014), 8, 462), peripheral blood mono-nuclear cells (PBMCs) were isolated by density centrifugation gradient using Ficoll-Paque Plus (GE Healthcare Biosciences AB, Uppsala, Sweden) from buffy coats provided by the National Transfusion Centre of the Comunidad Autonoma of Madrid, and splenocytes were obtained from C57/BL6 male mice. For carboxyfluorescein diacetate N-succinimidyl ester (CFSE) labelling, PBMCs or splenocytes were washed extensively to remove FBS, resuspended in a 10 μM CFSE (Sigma-Aldrich, St Louis, Mo., USA) solution (107 PBMC or splenocytes per 200 μl of solution), and incubated under constant shaking at 37° C. for 10 min. The reaction was stopped by adding ice-cold medium (RPMI+10% FBS), and cells were washed three times with ice-cold PBS. Cells were then cultured overnight, and one aliquot was used to set up and control the FL-1 voltage for CFSE. After resting overnight, CFSE-labelled PBMCs were activated with the Pan T Cell Activation Kit (microbeads coated with anti-CD3, anti-CD2, and anti-CD28; Miltenyi Biotec, Auburn, Calif., USA) following the manufacturer's instructions. CFSE-labelled splenocytes were activated with anti-CD3 (Becton Dickinson) and IL-2 (Novartis, Basel, Switzerland). PBMCs or splenocytes (1 million cells/well) were cultured in 24-well plates alone or with eASCs (4×10⁴ cells/well; ratio 1:25 of eASC:PBMC or eASC: splenocytes) in a total volume of 2 mL of RPMI+10% FBS. The ASC:PBMC ratio of 1:75 allowed assessment of differences between samples in sub-optimal conditions. After 5 days for PBMCs and 3 days for splenocytes, cells were harvested, labelled with 7-AAD and anti-CD3 antibody and cell proliferation of the CD3+/7-AAD—population (viable CD3 T lymphocytes) was determined by flow cytometry, according to loss of CFSE signal. The data were analysed using the FCSExpress 4 (De Novo Software, Glendale, Calif., USA) and BD CellQuest™ Pro analysis (Becton Dickinson) software. CaliBRITE beads (BD Bioscience, Erembodegem-Aalst, Belgium) were used to calibrate the acquisition events in the cytometer.

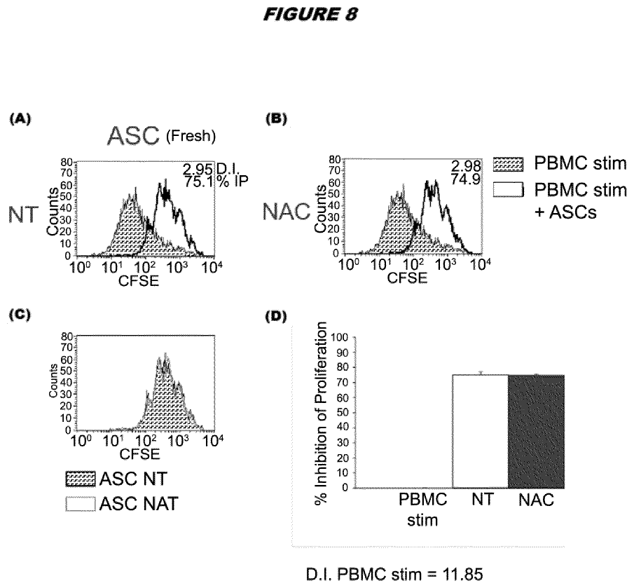

The inhibitory capacity of ASCs pre-treated with NAC prior to freezing was similar to non-treated cells (FIG. 8) (a slight tendency of NAC pretreatment to boost the inhibitory capacity of ASC was also observed in one or two experiments).

### Example 7—Assessment of NAC Pretreatment on the Effect of ASC on Macrophage and mDC Differentiation and Function

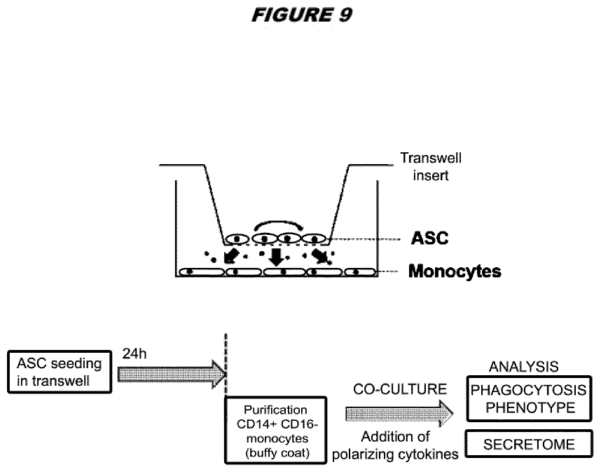

A second functional in vitro assay that was performed to assess the effect of NAC in the immunomodulatory capacity of ASC was the modulation of monocyte differentiation. FIG. 9 shows the timing and setup of the experiments.

### Blood Samples

Buffy Coats were obtained from the Transfusions Center at Comunidad de Madrid. About 50-60 mL of blood were diluted with PBS at room temperature and distributed between 50 mL tubes on top of 15 mL of room temperature Ficoll Hypaque Plus. Then the tubes were centrifuged for 40 minutes at 2000 rpm at 10° C. without brake or acceleration. The white ring of PBMCs were collected, washed in 50 mL of cold PBS and centrifuged for 15 minutes at 1800 rpm at 10° C. without brake or acceleration. After a second wash with 50 mL of cold RPMI complete medium (RPMIc: RPMI with 10% FBS, 2 mM L-Glu and 100 μg/m1Pen/Strep), the tubes were centrifuged for 15 minutes at 1500 rpm at 10° C. with brake and acceleration. A last wash was done in 50 mL of cold RPMIc and centrifuged for 15 minutes at 1200 rpm at 10° C. with brake and acceleration. PBMCs were resuspended in RPMIc and counted. Cells were resuspended at 100 million cells/mL in cold and the same volume of cold RPMIc supplemented with 10% DMSO was added, i.e. final concentration 5% DMSO. PBMCs were frozen in liquid nitrogen in vials of 50 million PBMCs.

### Isolation of CD14⁺ Monocytes

Frozen vials of PBMCs were thawed, counted and CD14⁺ CD16⁻ monocytes were isolated using the Dynabeads Untouched Human Monocytes kit (Dynal #11350D), following the manufacturer's instructions.

### Culture and Differentiation of Human Monocytes

Isolated CD14⁺ CD16⁻ monocytes (see above) were plated in 5 mL of RPMIc at 1.5 million cells per 6-well (Falcon #353046), in normoxia. The following factors were added for differentiation into non-polarized M0 macrophages or further polarization into M1, M2 macrophages and mature dendritic cells (mDC) populations in monocultures (based on several publications, including Beyer et al., PLoS One (2012) 7(9): e45466; Erbel et al., J. Vis. Exp. (2013) 76: e50332; Zhou et al, 2014; Tarique et al, American Journal of Respiratory Cell and Molecular Biology 2015; 53(5):676-688.

Immature DC (iDC): RPMIc+5 ng/mL GM-SCF+10 ng/mL IL-4 for 5 days
Maturation of DC (mDC): at day 5, add 40 ng/mL LPS (i.e. add 500 μL/well of RPMIc supplemented with 400 ng/mL LPS to the pre-existing media).
Human recombinant GM-CSF (#100-22B) and IL-4 (#200-04) were from Peprotech. LPS (#L8274) was from SIGMA. The addition of GM-C SF and IL4 mediates the differentiation to immature dendritic cells (iDC); 5 days later addition of LPS induces the maturation of iDCs to mDCs, and after 2 days the phenotype and function of these mature DC were analysed in the presence or absence of the ASC.
Co-Culture Experiments with ASCs

Freshly isolated human CD14⁺ CD16⁻ monocytes were co-cultured with ASCs from donor A or donor B in polycarbonate 6-well transwells (Corning #3412 inserts and Falcon #353046 plates).

NAC pretreated ASCs (according to the methods in Example 2), or non-treated, ASCs were thawed, and 150,000 ASCs were plated on the transwell inserts 16 hours or 24 hours prior to co-culture setting in 1 mL of RPMIc media, and 1.5 million monocytes were placed at the bottom of the well in 4 mL of RPMIc media. The differentiation was carried out using the same factors as in differentiation of monocytes alone (see above, namely addition of GM-C SF and IL4 to induce differentiation to iDC; 5 days later addition of LPS to induce the maturation of iDCs to mDCs, and after 2 days the phenotype and function of these mature DC were analysed in the presence or absence of the ASC). ASCs were kept in the transwell insert for the entire duration of the differentiation process.

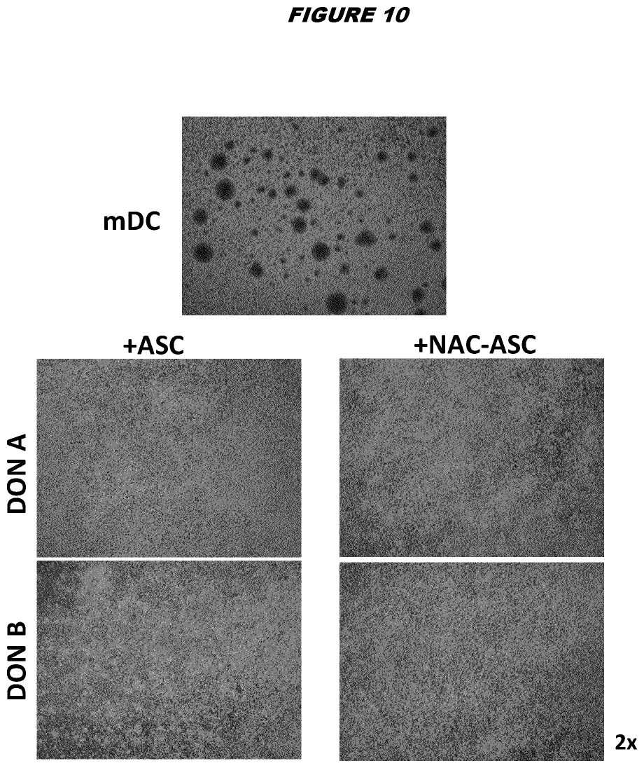

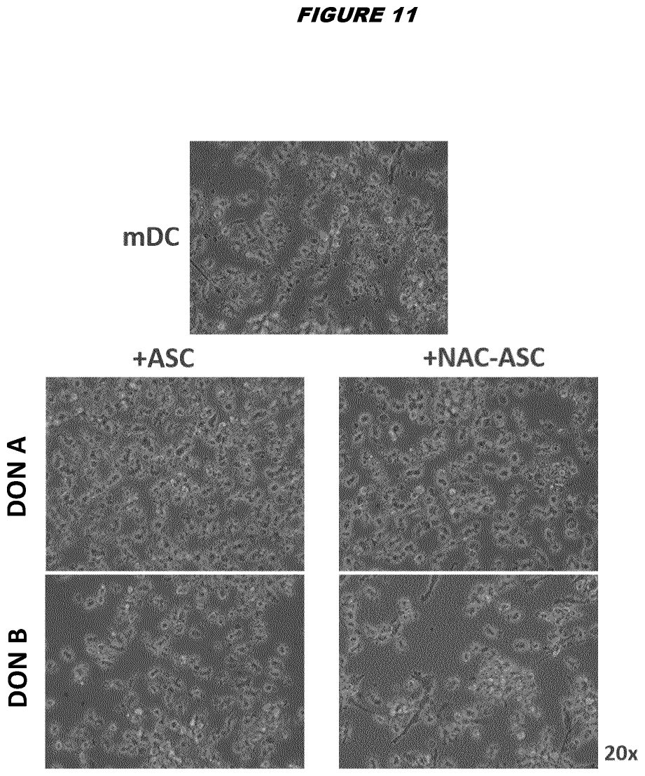

Activation clusters did not form on the plates after co-culture of the mDC with NAC pretreated or non-treated ASCs, indicating that the ASCs modulate the activation of mDCs and this effect is not disrupted by NAC pretreatment (see microscopy images as 2× magnification (FIG. 10) and 20× magnification (FIG. 11)).

### Example 8—NAC Pretreatment does not Significantly Alter the Ability of ASCs to Modulate the Phagocytosis of Staphylococcus aureus Particles by mDC

The effect of NAC pretreatment (according to the methods in Example 2) on the capacity of thawed ACS to modulate mDCs ability phagocytose Staphylococcus aureus particles was analysed.

After differentiation in monoculture or co-culture in vitro (the differentiation conditions, including the cytokines used, concentrations and times of differentiation, are provided in Example 7), macrophages and mDC were harvested using 0.05% Trypsin-EDTA for 10 minutes at 37° C. The phagocytic potential of polarized macrophages or mDC was assessed using pHRodo Red-conjugated S. aureus particles (Life Technologies #A10010) following manufacturer's instructions. Briefly, 50,000 mDC were transferred to a 96-well U bottom wells (Corning #3799) and the cells were rested for 60 minutes in RPMIc. Lyophilized pHRodo conjugated particles were reconstituted in 1 mL of RPMIc per vial prior to use, and particles were sonicated for 5 minutes at 20% amplitude. Then, 50 μL of pHRodo Zymosan were added per well and the cells were incubated for 60 minutes in normoxia at 37° C. Afterwards, phagocytosis was stopped on ice and cells were washed and stained with 5 μL 7-aminoactinomycin D (7AAD) prior to FACS analysis in Fortessa cytometer (BD). Negative controls for phagocytosis were cells without pHRodo reagent. Results were analysed in FlowJo software. The intensity of the fluorescence in the PE channel is proportional to the amount of bacterial particles phagocytosed per cell.

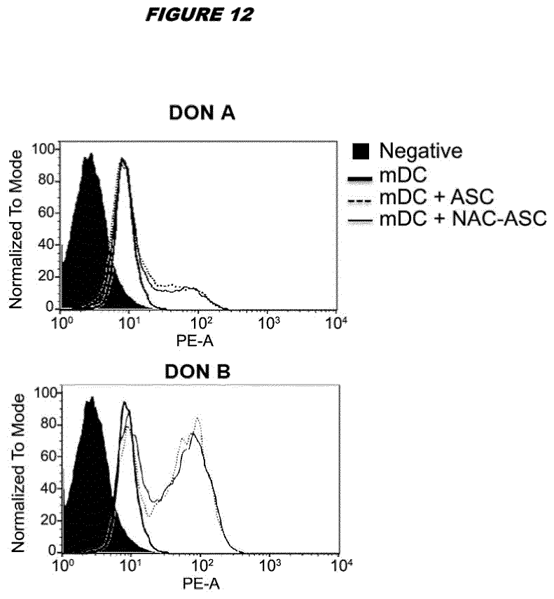

FIG. 12 shows that the presence of ASC in non-contact conditions (i.e. the mDC and ASC cells were co-cultured in transwell plates) results in the appearance of a new population of cells that is more intense in the fluorescence channel, i.e. cells that have phagocytosed fluorescent particles. NAC pre-treatment of ASC with NAC did not alter their ability to increase the phagocytosis potential of mDCs.

### Example 9—Effect of NAC Pretreatment on ASC Mediated Effects on the Surface Expression on Mature Dendritic Cells

The capacity of mDC to phagocytose bacteria is linked to the expression of phagocytic markers, such as CD209 (DC-SIGN), CD206 (mannose receptor) or CD163 (scavenger receptor). These membrane receptors recognise specific patterns on the surface of fungi, bacteria and parasites and mediate their phagocytosis by monocytes, macrophages and DC. The CD163 receptor additionally intervenes in the clearance of cell debris from apoptotic cells after tissue damage, contributing to the process of wound healing.

The effect of NAC pretreatment (according to Example 2) on thawed ASC mediated effects on the surface expression of the phagocytic receptors CD206 (mannose receptor) and CD163 (scavenger receptor) by mature dendritic cells was measured by flow cytometry.

### Phenotypic Characterization

After differentiation in monoculture or co-culture in vitro (the differentiation conditions, including the cytokines used, concentrations and times of differentiation, are provided in Example 7), macrophages and mDC were harvested using 0.05% trypsin-EDTA for 10 minutes at 37° C., after supernatants were collected and frozen for future cytokine and/or HPLC analysis. After mDC were counted, they were distributed into 96-well V bottom plates for staining (Nunc #249570). Cells were incubated in Blue MACS buffer with 1% human serum for 15 minutes on ice, to block Fcγ receptor-mediated nonspecific antibody binding. Subsequently, cells were stained for 20 minutes on ice with the following antibody mixes (staining in 50 μL of 1:10 antibody dilution; except for CD64, which was 1:20 dilution):

| key | staining (ALL WELLS WITH 7AAD) |
|---|---|
| 1 | CD14-APC/HLAII-FITC 1:50/CD86-PE |
| 2 | CD14-APC/CD206-PE/CD209-FITC |
| 3 | CD14-APC/CD163-PE |
| 4 | CD14-APC/CD80-FITC/CD64-PE 1:20 |
| 5 | CD14-APC/CD1a-PE |

The details of the antibodies used are listed in the following table:

| NAME | FLUOROCROME | HOST | CLONE | CAT. NUMBER | COMPANY |
|---|---|---|---|---|---|
| CD1a | PE | MOUSE | HI149 | 555807 | BD |
| CD14 | APC | MOUSE | M5E1 | 555399 | BD |
| CD64 | PE | MOUSE | 10.1 | CD6404 | Miltenyi Biotech |
| CD68 | PeCy7 | MOUSE | 27-35 | 560542 | BD |
| CD80 | PE | MOUSE | L307.4 | 557227 | BD |
| CD86 | PE | MOUSE | IT2.2 | 555665 | BD |
| CD206 | PE | MOUSE | 19.2 | 555954 | BD |
| CD209 | FITC | MOUSE | DCN46 | 551264 | BD |
| HLA-II | PE | MOUSE | WR18 | MA1-80680 | Ebiosciences |

Cell viability was assessed by addition of 5 μL of 7AAD per well and staining for 10 minutes on ice, and samples were acquired in a BD Fortessa cytometer. Results were analysed in FSC Express software.

The capacity of mDC to phagocytose bacteria is linked to the expression of phagocytic markers, such as CD209 (DC-SIGN), CD206 (mannose receptor) or CD163 (scavenger receptor). These membrane receptors recognise specific patterns on the surface of fungi, bacteria and parasites and mediate their phagocytosis by monocytes, macrophages and DC. The CD163 receptor additionally intervenes in the clearance of cell debris from apoptotic cells after tissue damage, contributing to the process of wound healing. The effect of NAC pretreatment (according to Example 2) on thawed ASC mediated effects on the surface expression of the phagocytic receptors CD206 (mannose receptor) and CD163 (scavenger receptor) by mature dendritic cells was measured by flow cytometry.

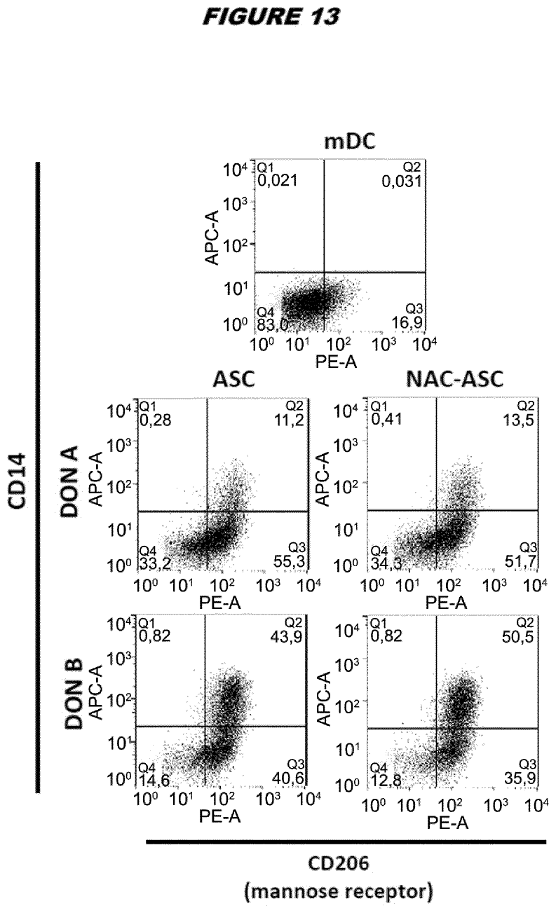

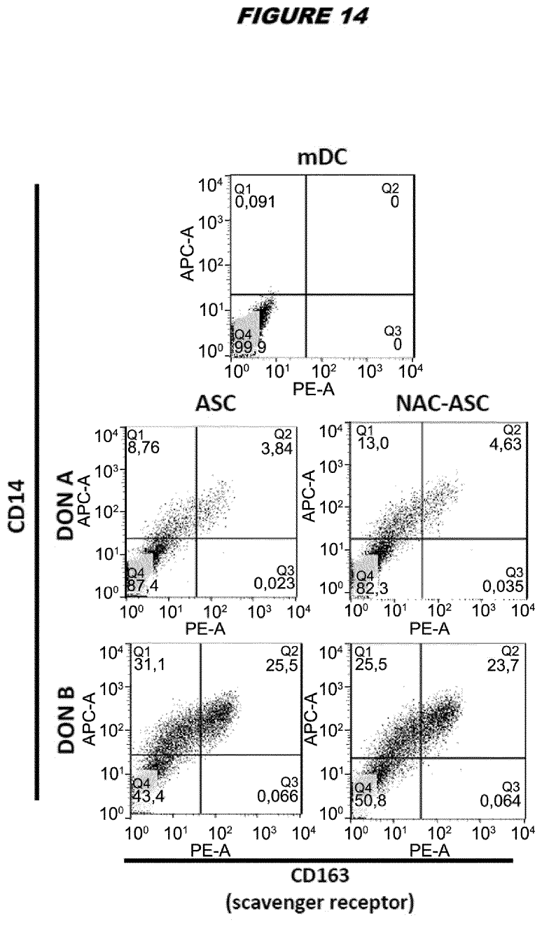

ASC upregulate the expression of CD206 and CD163 markers on the surface of monocytes, macrophages and mDC, and this upregulation was intact even when ASC were pre-treated with NAC (FIGS. 13 and 14).

The effect of NAC pretreatment (according to Example 2) on thawed ASC mediated effects on the surface expression of CD14 and CD1a on mature dendritic cells was also measured by flow cytometry. mDC are CD14-CD1a+. CD1a is the antigen-presenting molecule, and mediates the presentation of antigens by mDC to other cells of the immune system to activate their response. ASC modulate the phenotype of these mDC, turning them into CD14+CD1a− cells. This population has been attributed anti-inflammatory and regulatory properties (Chang et al., Journal of Immunology, 165(7), 3584-3591).

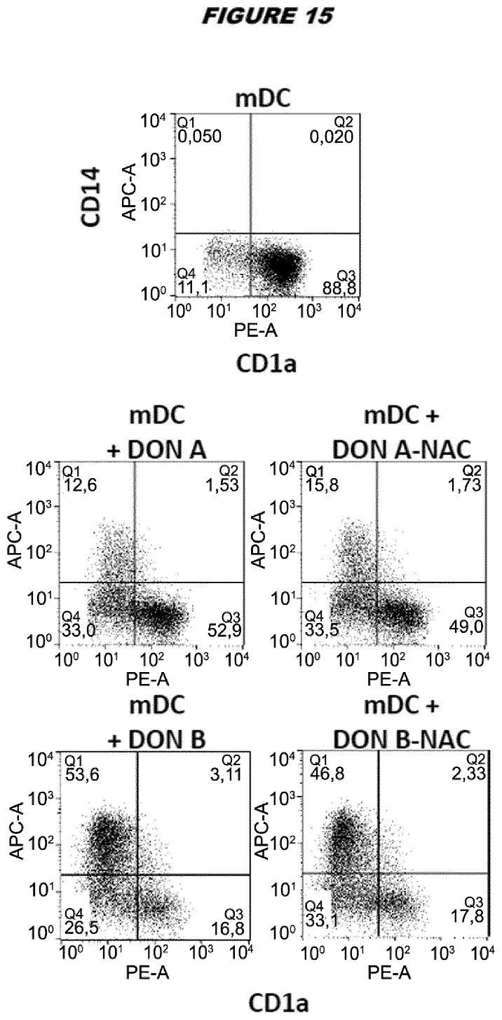

FIG. 15 shows that NAC pretreatment of ASC does not alter the ability of thawed ASCs to induce the formation of this mDC regulatory population.

### Numbered Embodiments

The invention also provides the following numbered embodiments:
1. A method for stem cell cryopreservation, the method comprising the steps of:
   a. treating a population of stem cells with N-acetylcysteine (NAC) to obtain a treated population of stem cells; and
   b. freezing the treated population of stem cells to obtain a frozen population of stem cells.
2. The method of embodiment 1, wherein the method comprises the steps of:
   a. treating the population of stem cells with NAC to obtain a treated population of stem cells;
   b. freezing the treated population of stem cells to obtain a frozen population of stem cells; and
   c. thawing the frozen population of stem cells to obtain a thawed population of stem cells.
3. The method of embodiment 1 or embodiment 2, wherein the method comprises the steps of:
   a. treating the population of stem cells with NAC to obtain a treated population of stem cells;
   b. washing the treated population of stem cells to remove the NAC and to obtain a washed population of stem cells, and freezing the washed population of stem cells to obtain a frozen population of stem cells; and
   c. thawing the frozen population of stem cells to obtain a thawed population of stem cells.
4. The method of any one of the preceding embodiments, wherein the treatment step comprises incubating the population of stem cells with NAC for at least about 1, 2, 4, 6, 8, 10, 12, 16, 24 or 48 hours prior to freezing the population of stem cells.
5. The method of any one of the preceding embodiments, wherein the treatment step comprises adding NAC to the population of stem cells to an initial concentration in the range of around 0.5-10 mM.
6. The method of embodiment 5, wherein the treatment step comprises one or more further additions of NAC to maintain the concentration of NAC at a preselected level.
7. The method of any one of embodiments 2-6, wherein the method further comprises the step of:
   d. culturing the thawed population of stem cells to obtain an expanded population of stem cells.
8. The method of any one of embodiments 2-6, wherein the method further comprises the step of:
   d. culturing the thawed population of stem cells in the presence NAC to obtain an expanded population of stem cells.
9. The method of embodiment 8, wherein the culturing step comprises adding NAC to an initial concentration in the range of around 0.5-5 mM.
10. The method of embodiment 9, wherein the culturing step comprises one or more further additions of NAC to maintain the concentration of NAC at a preselected level.
11. The method of any one of embodiments 8-10, wherein the method further comprises a step of washing the expanded population of stem cells to remove the NAC and to obtain a washed and expanded population of stem cells.
12. The method of any one of embodiments 2-11, wherein the method further comprises a step of washing the thawed population of stem cells or the expanded population of stem cells and resuspending the cells in a pharmaceutically acceptable carrier.
13. The method of any one of embodiments 7-12, wherein the method further comprises the step of:
   e. freezing the expanded or the washed and expanded population of stem cells to obtain a frozen expanded population of stem cells or a frozen, washed and expanded population of stem cells.
14. The method of any one of embodiments 7-13, wherein the method further comprises the steps of:
   e. freezing the expanded or the washed and expanded population of stem cells to obtain a frozen expanded population of stem cells or a frozen, washed and expanded population of stem cells; and
   f. thawing the frozen expanded or the frozen, washed and expanded population of stem cells to obtain a thawed expanded population of stem cells.
15. The method of embodiment 14, wherein the method further comprises the step of:
   g. washing the thawed expanded population of stem cells and resuspending the cells in a pharmaceutically acceptable carrier.
16. A method for stem cell cryopreservation, the method comprising the steps of:
   a. freezing a population of stem cells to obtain a frozen population of stem cells;
   b. thawing the frozen population of stem cells to obtain a thawed population of stem cells; and
   c. culturing the thawed population of stem cells in the presence NAC to obtain an expanded population of stem cells.
17. The method of embodiment 16, wherein the culturing step comprises adding NAC to an initial concentration of around 0.5-5 mM.
18. The method of embodiment 17, wherein the culturing step comprises one or more further additions of NAC to maintain the concentration of NAC at a preselected level.
19. The method of any one of the preceding embodiments, wherein the freezing step comprises reducing the temperature to between −70° C. and −130° C. at a rate of between about −0.5 to about −10° C./minute.
20. The method of any one of the preceding embodiments, wherein the freezing step comprises reducing the temperature from +4° C. to between −100 and −180° C. in 10-60 mins.
21. The method of any one of the preceding embodiments, wherein the population of stem cells is thawed at 37° C.
22. The method of any one of the preceding embodiments, wherein the cell density of frozen population of stem cells is in the range of around 1 million to around 50 million cells/mL, preferably around 25 million cells/mL.
23. The method of any one of the preceding embodiments, wherein the population of stem cells is substantially pure.
24. The method of any one of the preceding embodiments, wherein the stem cells are mesenchymal stem cells (MSCs).
25. The method of any one of the preceding embodiments, wherein the stem cells are adipose-derived stromal stem cells (ASCs).
26. The method of any one of the preceding embodiments, wherein the stem cells are human cells.
27. The method of any one of the preceding embodiments, wherein the method further comprises the step of resuspending the cells in a pharmaceutically acceptable carrier.
28. The method of any one of the preceding embodiments, wherein the method comprises freezing the population of stem cells in a plurality of cryovials.
29. The method of any one of the preceding embodiments, wherein the method comprises repeating the steps of any one of the preceding embodiments for a plurality of populations of stem cells.
30. The method of embodiment 29, wherein the method comprises freezing the plurality of populations of stem cells in a plurality of cryovials.
31. The method of embodiment 28 or embodiment 30, wherein the method comprises storing the plurality of cryopreservation vials in a liquid nitrogen storage container for at least one month at least 2 months, at least 3 months, at least 6 months, or at least 1 year.
32. A liquid nitrogen storage container containing the plurality of cryopreservation vials obtained according to the method of embodiment 28 or embodiment 30.
33. A population of stem cells obtained by the method of any one of embodiments 1-31.
34. The method of any one of embodiments 1-31 or the population of stem cells of embodiment 33, wherein the number of viable cells following thaw and optionally culture for about 1 day or about 4 days is increased as compared to a control population of stem cells.
35. The method of any one of embodiments 1-31 and 34 or the population of stem cells of embodiment 33 or 34, wherein the number of viable cells following thaw is increased at least about 1.05-fold, at least about 1.1-fold, at least about 1.2-fold, at least about 1.3-fold, at least about 1.4-fold, at least about 1.5-fold, at least about 1.6-fold, at least about 2-fold, or at least about 5-fold as compared to a control population of stem cells.
36. The method of any one of embodiments 1-31, 34 or 35 or the population of stem cells of embodiment 33-35, wherein the growth rate following thaw is increased at least about at least about 1.03-fold, 1.05-fold, at least about 1.1-fold, at least about 1.15-fold, at least about 1.2-fold, at least about 1.25-fold, at least about 1.3-fold, at least about 1.4-fold, at least about 1.6-fold, or at least about 2-fold in the population of stem cells as compared to a control population of stem cells.
37. The method of any one of embodiments 1-31, 34-36 or the population of stem cells of embodiment 33-36, wherein mitochondrial activity following thaw and optionally culture for about 1 day or about 4 days is increased at least about 5%, at least about 10%, at least about 15%, at least about 20%, at least about 30%, at least about 35%, at least about 40% or at least about 50% as compared to a control population of stem cells.
38. The method of any one of embodiments 1-31, 34-37 or the population of stem cells of embodiment 33-37, wherein the time taken post-thaw for the ASCs to recover is decreased as compared to a control population of stem cells.
39. The method of any one of embodiments 1-31, 34-38 or the population of stem cells of embodiment 33-38, wherein the number of hours taken for the cells to recover post-thaw is decreased at least about 1.1-fold, at least about 1.2-fold, at least about 1.4-fold, at least about 1.6-fold, at least about 2-fold, at least about 3-fold, at least about 4-fold, or at least about 5-fold relative to a control population of stem cells.
40. A cryopreservation composition comprising the population of stem cells of any one of embodiments 33-38 and a cryopreservation medium.
41. The cryopreservation composition of embodiment 40, wherein the composition is frozen.
42. The cryopreservation composition of embodiment 40 or embodiment 41, wherein the composition contains NAC.
43. A pharmaceutical composition comprising the population of stem cells of any one of embodiment 33-38 and a pharmaceutically acceptable carrier.
44. The pharmaceutical composition of embodiment 43, where the composition comprises around 1 million cells to around 150 million cells, preferably around 30 million cells or around 120 million cells.
45. The pharmaceutical composition of embodiment 43 or embodiment 44, where the cell density is around 1 to 20 million cells/mL.
46. Use of NAC for the cryopreservation of stem cells.
47. The use of NAC according to embodiment 46 in the method of any one of embodiments 1-31 and 34-39.
48. The population of stem cells of any one of embodiments 33-39, pharmaceutical composition of any one of embodiments 43-45 or cryopreservation composition of embodiment 40-42 for use in therapy.
49. The population of stem cells of any one of embodiments 33-39, pharmaceutical composition of any one of embodiments 43-45 or cryopreservation composition of embodiment 40-42 for use in a method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection in a patient in need thereof.
50. A method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection, the method comprising administering the population of stem cells of any one of embodiments 33-39, pharmaceutical composition of any one of embodiments 43-45 or cryopreservation composition of embodiment 40-42 to a subject in need thereof.
51. A population of stem cells for use in a method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection, in a patient in need thereof, wherein the method comprises the steps of:
   a. treating of a population of stem cells with NAC to obtain a treated population of stem cells;
   b. freezing the treated population of stem cells to obtain a frozen population of stem cells;
   c. thawing the frozen population of stem cells to obtain a thawed population of stem cells;
   d. optionally culturing the thawed population of stem cells to obtain an expanded population of stem cells;
      and
   e. administering the population of stem cells to the patient.
52. A method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection, in a patient in need thereof, the method comprising the steps of:
   a. treating a population of stem cells with NAC to obtain a treated population of stem cells;
   b. freezing the treated population of stem cells to obtain a frozen population of stem cells;
   c. thawing the frozen population of stem cells to obtain a thawed population of stem cells;
   d. optionally culturing the thawed population of stem cells to obtain an expanded population of stem cells;
      and
   e. administering the population of stem cells to the patient.
53. The population of stem cells for use according to embodiment 51 or method of treatment of embodiment 52, wherein the method further comprises any one of the steps as defined in embodiments 3-14, 18-31 or 34-39 prior to administration of the population of stem cells to the patient.
54. A population of stem cells for use in a method of treating fistula and/or treating and/or preventing an inflammatory disorder, an autoimmune disease or an immunologically-mediated disease, such as sepsis, rheumatoid arthritis, allergies (e.g. hypersensitivity Type IV reactions), irritable bowel disease, Crohn's disease, ulcerative colitis or organ rejection in a patient in need thereof, wherein the method comprises the steps of:
   a. freezing a population of stem cells to obtain a frozen population of stem cells;
   b. thawing the frozen population of stem cells to obtain a thawed population of stem cells;
   c. culturing the thawed population of stem cells in the presence NAC to obtain an expanded population of stem cells; and
   d. administering the population of stem cells to the patient.
55. A method of treating fistula and/or treating and/or preventing an inflammatory disorder, an autoimmune disease, or an immunologically-mediated disease, such as sepsis, rheumatoid arthritis, allergies (e.g. hypersensitivity Type IV reactions), irritable bowel disease, Crohn's disease, ulcerative colitis or organ rejection, in a patient in need thereof, the method comprising the steps of:
   a. freezing a population of stem cells to obtain a frozen population of stem cells;
   b. thawing the frozen population of stem cells to obtain a thawed population of stem cells;
   c. culturing the thawed population of stem cells in the presence NAC to obtain an expanded population of stem cells; and
   d. administering the population of stem cells to the patient.
56. The population of stem cells for use according to embodiment 54 or method of treatment of embodiment 55, wherein the method further comprises any one of the steps as defined in any one of embodiments 15-31 or 34-39 prior to administration of the population of stem cells to the patient.
57. The population of stem cells, pharmaceutical composition or cryopreservation composition for use according to any one of embodiments 48, 49, 51, 53, 54 or 56, or the method of any one of embodiments 50, 52, 53, 55 or 56, wherein the method comprises administering around 1 million to 150 million cells, preferably around 30 million stem cells or around 120 million stem cells.
58. The population of stem cells, pharmaceutical composition or cryopreservation composition for use according to any one of embodiments 48, 49, 51, 53, 54, 56 or 57, or the method of any one of embodiments 50, 52, 53, 55-57, wherein the method comprises administering around 1 million to around 10 million cells/kg.
59. The population of stem cells or pharmaceutical composition or cryopreservation composition for use according to any one of embodiments 48, 49, 51, 53, 54, 56-58, or the method of any one of embodiments 50, 52, 53, 55-58, wherein the method comprises injecting the population of stem cells or pharmaceutical composition of any one of embodiments 43-45 or cryopreservation composition of any one of embodiments 40-42.
60. The population of stem cells or pharmaceutical composition or cryopreservation composition for use according to any one of embodiments 48, 49, 51, 53, 54, 56-59, or the method of any one of embodiments 50, 52, 53, 55-59, wherein the stem cells are as defined in any one of embodiments 23-26.
61. The population of stem cells or pharmaceutical composition or cryopreservation composition for use according to any one of embodiments 48, 49, 51, 53, 54, 56-60, or the method of any one of embodiments 50, 52, 53, 55-60, wherein the stem cells are allogeneic or autologous.
62. A cryopreservation kit comprising: a cryovial, a container containing NAC and a container comprising a population of stem cells.

1. A method for stem cell cryopreservation, the method comprising the steps of:
a. treating a population of stem cells with N-acetylcysteine (NAC) to obtain a treated population of stem cells; and
b. freezing the treated population of stem cells to obtain a frozen population of stem cells.

2. The method of claim 1, wherein the method comprises the steps of:
a. treating the population of stem cells with NAC to obtain a treated population of stem cells;
b. freezing the treated population of stem cells to obtain a frozen population of stem cells; and
c. thawing the frozen population of stem cells to obtain a thawed population of stem cells.

3. The method of claim 1 or claim 2, wherein the method comprises the steps of:
a. treating the population of stem cells with NAC to obtain a treated population of stem cells;
b. washing the treated population of stem cells to remove the NAC and to obtain a washed population of stem cells, and freezing the washed population of stem cells to obtain a frozen population of stem cells; and
c. thawing the frozen population of stem cells to obtain a thawed population of stem cells.

4. The method of any one of the preceding claims, wherein the treatment step comprises:
incubating the population of stem cells with NAC for at least about 1, 2, 4, 6, 8, 10, 12, 16, 24 or 48 hours prior to freezing the population of stem cells; and/or
adding NAC to the population of stem cells to an initial concentration in the range of around 0.5-10 mM, optionally wherein the treatment step comprises one or more further additions of NAC to maintain the concentration of NAC at a preselected level.

5. The method of any one of claims 2-4, wherein the method further comprises the step of:
d. culturing the thawed population of stem cells to obtain an expanded population of stem cells.

6. The method of any one of claims 2-4, wherein the method further comprises the step of:
d. culturing the thawed population of stem cells in the presence NAC to obtain an expanded population of stem cells, optionally wherein:
the culturing step comprises adding NAC to an initial concentration in the range of around 0.5-5 mM, further optionally wherein the culturing step comprises one or more further additions of NAC to maintain the concentration of NAC at a preselected level; and/or
the method further comprises a step of washing the expanded population of stem cells to remove the NAC and to obtain a washed and expanded population of stem cells.

7. The method of any one of claims 2-6, wherein the method further comprises a step of washing the thawed population of stem cells or the expanded population of stem cells and resuspending the cells in a pharmaceutically acceptable carrier.

8. The method of any one of claims 6-7, wherein the method further comprises the step of:
e. freezing the expanded or the washed and expanded population of stem cells to obtain a frozen expanded population of stem cells or a frozen, washed and expanded population of stem cells; and optionally
f. thawing the frozen expanded or the frozen, washed and expanded population of stem cells to obtain a thawed expanded population of stem cells; and further optionally
g. washing the thawed expanded population of stem cells and resuspending the cells in a pharmaceutically acceptable carrier.

9. A method for stem cell cryopreservation, the method comprising the steps of:
a. freezing a population of stem cells to obtain a frozen population of stem cells;
b. thawing the frozen population of stem cells to obtain a thawed population of stem cells; and
c. culturing the thawed population of stem cells in the presence NAC to obtain an expanded population of stem cells, optionally wherein the culturing step comprises adding NAC to an initial concentration of around 0.5-5 mM, further optionally wherein the culturing step comprises one or more further additions of NAC to maintain the concentration of NAC at a preselected level.

10. The method of any one of the preceding claims, wherein the stem cells are mesenchymal stem cells (MSCs) and/or wherein the stem cells are adipose-derived stromal stem cells (ASCs).

11. A population of stem cells obtained by the method of any one of claims 1-10.

12. The population of stem cells of claim 11, wherein:
the number of viable cells following thaw and optionally culture for about 1 day and/or about 4 days is increased as compared to a control population of stem cells;
the number of viable cells following thaw is increased at least about 1.05-fold, at least about 1.1-fold, at least about 1.2-fold, at least about 1.3-fold, at least about 1.4-fold, at least about 1.5-fold, at least about 1.6-fold, at least about 2-fold, or at least about 5-fold as compared to a control population of stem cells;
the growth rate following thaw is increased at least about at least about 1.03-fold, 1.05-fold, at least about 1.1-fold, at least about 1.15-fold, at least about 1.2-fold, at least about 1.25-fold, at least about 1.3-fold, at least about 1.4-fold, at least about 1.6-fold, or at least about 2-fold in the population of stem cells as compared to a control population of stem cells;
mitochondrial activity following thaw and optionally culture for about 1 day and/or about 4 days is increased at least about 5%, at least about 10%, at least about 15%, at least about 20%, at least about 30%, at least about 35%, at least about 40% or at least about 50% as compared to a control population of stem cells;
the time taken post-thaw for the ASCs to recover is decreased as compared to a control population of stem cells; and/or
the number of hours taken for the cells to recover post-thaw is decreased at least about 1.1-fold, at least about 1.2-fold, at least about 1.4-fold, at least about 1.6-fold, at least about 2-fold, at least about 3-fold, at least about 4-fold, or at least about 5-fold relative to a control population of stem cells,
wherein the control population of stem cells is derived from the same population of stem cells as the population of stem cells treated with NAC and has not been treated with NAC, but has otherwise been subjected to identical conditions.

13. The method of any one of claims 1-10, wherein:
the number of viable cells following thaw and optionally culture for about 1 day and/or about 4 days is increased as compared to a control population of stem cells;
the number of viable cells following thaw is increased at least about 1.05-fold, at least about 1.1-fold, at least about 1.2-fold, at least about 1.3-fold, at least about 1.4-fold, at least about 1.5-fold, at least about 1.6-fold, at least about 2-fold, or at least about 5-fold as compared to a control population of stem cells;
the growth rate following thaw is increased at least about at least about 1.03-fold, 1.05-fold, at least about 1.1-fold, at least about 1.15-fold, at least about 1.2-fold, at least about 1.25-fold, at least about 1.3-fold, at least about 1.4-fold, at least about 1.6-fold, or at least about 2-fold in the population of stem cells as compared to a control population of stem cells;
mitochondrial activity following thaw and optionally culture for about 1 day and/or about 4 days is increased at least about 5%, at least about 10%, at least about 15%, at least about 20%, at least about 30%, at least about 35%, at least about 40% or at least about 50% as compared to a control population of stem cells;
the time taken post-thaw for the ASCs to recover is decreased as compared to a control population of stem cells; and/or
the number of hours taken for the cells to recover post-thaw is decreased at least about 1.1-fold, at least about 1.2-fold, at least about 1.4-fold, at least about 1.6-fold, at least about 2-fold, at least about 3-fold, at least about 4-fold, or at least about 5-fold relative to a control population of stem cells.

14. A cryopreservation composition comprising the population of stem cells of claim 11 or claim 12 and a cryopreservation medium, optionally wherein the composition is frozen and/or optionally wherein the composition contains NAC.

15. Use of NAC for the cryopreservation of stem cells.

16. A cryopreservation kit comprising: a cryovial, a container containing NAC and a container comprising a population of stem cells.
